SEQUENCE LISTING (1 sequence)

SEQ ID NO: 1 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVDVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

(19) United States
(12) Patent Application Publication
(10) Pub. No.: US 2021/0400894 A1
(43) Pub. Date: Dec. 30, 2021

(54) Plants with a Cannabinoid Profile Enriched for Cannabidiol

(71) Applicant: Agriculture Victoria Services Pty Ltd, Bundoora (AU)

(72) Inventors: Aaron Christopher Elkins, Taylors Hill (AU); Simone Jane Rochfort, Reservoir (AU); Noel Cogan, Macleod (AU); German Carlos Spangenberg, Bundoora (AU)

(21) Appl. No.: 17/292,199

(22) PCT Filed: Nov. 8, 2019
(86) PCT No.: PCT/AU2019/051229

§ 371 Date: May 7, 2021

(30) Foreign Application Priority Data
Nov. 9, 2018 (AU) 2018904285
Nov. 9, 2018 (AU) 2018904286
Nov. 9, 2018 (AU) 2018904289
Nov. 9, 2018 (AU) 2018904291
Jan. 31, 2019 (AU) 2019900291
Jan. 31, 2019 (AU) 2019900293
Jan. 31, 2019 (AU) 2019900294
Jan. 31, 2019 (AU) 2019900295

(51) Int. Cl. A01H 6/28; A01H 5/02; A61K 36/185

(52) U.S. Cl. CPC A01H 6/28; A01H 5/02; A61K 36/185; A61K 2236/37; A61K 2236/17; A61K 2236/33; A61K 2236/15

(57) ABSTRACT

The present disclosure relates generally to new Cannabis plants, including parts, extracts and uses thereof, comprising a cannabinoid profile enriched for total CBD (i.e., cannabidiol (CBD) and cannabidiolic acid (CBDA)).

The present application claims priority from Australian Provisional Patent Applications 2018904285, 2018904286, 2018904289 and 2018904291 filed 9 Nov. 2018 and Australian Provisional Patent Applications 2019900291, 2019900293, 2019900294 and 2019900295 filed 31 Jan. 2019, the disclosures of which are hereby expressly incorporated herein by reference in their entirety.

### FIELD

The present disclosure relates generally to new Cannabis plants, including parts, extracts and uses thereof, comprising a cannabinoid profile enriched for total CBD (i.e., cannabidiol (CBD) and cannabidiolic acid (CBDA)).

### BACKGROUND

Cannabis is an herbaceous flowing plant of the Cannabis genus (Rosale), which has been used for its fibre and medicinal properties for thousands of years. The medicinal qualities of Cannabis have been recognised since at least 2800 BC, with use of Cannabis featuring in ancient Chinese and Indian medical texts. Although use of Cannabis for medicinal purposes has been known for centuries, research into the pharmacological properties of the plant has been limited due to its illegal status in most jurisdictions.

The chemical profile of Cannabis plants is varied. It is estimated that Cannabis plants produce more than 400 different molecules, including phytocannabionids, terpenes and phenolics. Cannabinoids, such as Δ-9-tetrahydrocannabinol (THC) and cannabidiol (CBD), are typically the most commonly known and researched cannabinoids. CBD and THC are naturally present in their acidic forms, Δ-9-tetrahydrocannabinolic acid (THCA) and cannabidiolic acid (CBDA), which are alternative products of a shared precursor, cannabigerolic acid (CBGA).

Many cannabinoids interact with the endocannabinoid system in mammals, including humans, to exert complex biological effects on the neuronal, metabolic, immune and reproductive systems. They also interact with G protein-coupled receptors (GPCRs), such as CB1 and CB2, in the human endocannabinoid system, where they are thought to play a part in the regulation of appetite, pain, mood, memory, inflammation and insulin sensitivity. Cannabinoids have also been implicated in neuronal signaling, gastrointestinal inflammation, tumorigenesis, microbial infection and diabetes.

Whilst there is an increasing body of evidence of the therapeutic potential of Cannabis and Cannabis-derived compounds, in particular cannabinoids, their adoption into clinical practice has been hindered, at least in part, to the fact that their mechanisms of action remain largely ill-defined, noting also that different cannabinoids can exert different biological effects. The therapeutic potential of Cannabis and Cannabis-derived cannabinoids is further complicated by the entourage effect, where different cannabinoids act in combination to exert different biological effects. In view of this complexity, it is advantageous to select for new Cannabis varieties that have a cannabinoid profile enriched for specific cannabinoids suitable for therapeutic use.

Previous studies of the cannabinoid content of Cannabis plants have largely focused on the differentiation of Cannabis varieties bred for recreational or industrial use. For example, in a study conducted by Turner et al. (1979, Journal of Natural Products, 42:319-21), leaf material from 85 Cannabis varieties was screened for cannabichromene (CBC), CBD and THC in order to differentiate between recreational and industrial Cannabis varieties. The recreational varieties were subjected to further cannabinoid testing to identify the correct time for sampling due to the significant variation of cannabinoid biosynthesis over the life of the plan. More recently, nuclear magnetic resonance (NMR) spectroscopy and RT-PCR analysis has been used to investigate the metabolome and cannabinoid biosynthesis in the trichomes of Cannabis sativa “Bebiol” (Happyana and Kayser, 2016, Planta Medica, 82:1217-23). NMR metabolomics approaches have also been used to investigate the difference across 12 different Cannabis varieties using leaf and flower material (Choi et al. 2004, Journal of Natural Products, 67: 953-7). Applying principal component analysis to these results, it has been shown that the major discriminators of these varieties was THCA and CBDA, although carbohydrate and amino acid levels were also important discriminators for quality control and authentication purposes.

Despite these recent advances, there has been lack of sufficient systematic analysis for the purpose of precision breeding of Cannabis plants for medicinal use. There remains, therefore, an urgent need for systematic breeding and selection of improved Cannabis varieties comprising a cannabinoid profile enriched for specific cannabinoids that make them suitable for therapeutic use.

### SUMMARY

In an aspect disclosed herein, there is provided a Cannabis plant, or a part thereof, comprising a cannabinoid profile enriched for total CBD, wherein the cannabinoid profile comprises a level of total CBD and a level of total THC at a ratio of from about 10:1 to about 50:1 (CBD:THC); wherein the total CBD comprises cannabidiol (CBD) and cannabidiolic acid (CBDA), and the total THC comprises Δ-9-tetrahydrocannabinol (THC) and Δ-9-tetrahydrocannabinolic acid (THCA); wherein the level of total CBD is greater than the level of a reference cannabinoid selected from the group consisting of:
(a) total CBC, wherein the total CBC comprises cannabichromene (CBC) and cannabichromene acid (CBCA);
(b) total CBG, wherein the total CBG comprises cannabigerol (CBG) and cannabigerolic acid (CBGA);
(c) total CBN, wherein the total CBN comprises cannabinol (CBN) and cannabinolic acid (CBNA);
(d) total THCV, wherein the total THCV comprises tetrahydrocannabivarin (THCV) and tetrahydrocannabivarinic acid (THCVA); and
(e) total CBDV, wherein the total CBDV comprises cannabidivarin (CBDV) and cannabidivarinic acid (CBDVA),
   and wherein the Cannabis plant comprises a nucleic acid sequence that encodes a wild-type THCA synthase.

The present disclosure also extends to seeds produced from the Cannabis plant, and progeny plants derived therefrom.

In another aspect disclosed herein, there is provided a tissue culture of regenerable cells derived from the Cannabis plant as described herein, and progeny plants derived therefrom. In an embodiment, the progeny plant expresses the morphological and physiological characteristics of the Cannabis plant as described herein.

In another aspect disclosed herein, there is provided a method for producing an F1 hybrid Cannabis plant using plant breeding techniques which employ the Cannabis plant described herein, or a part thereof, as a source of plant breeding material. The present disclosure also extends to progeny plants and seeds produced from an F1 hybrid Cannabis plant, as described herein.

In another aspect disclosed herein, there is provided a method for producing a transgenic Cannabis plant, the method comprising transfecting the Cannabis plant described herein, or a part thereof, with a heterologous nucleic acid sequence to introduce one or more nucleic acid substitutions, deletions or additions into the genome of the Cannabis plant as described above. The present disclosure also extends to progeny plants and plant parts such as seeds produced from a transgenic Cannabis plant resulting from the methods disclosed herein.

In another aspect disclosed herein, there is provided a method of producing an extract comprising cannabinoids from a Cannabis plant, the method comprising harvesting plant material from the Cannabis plant described herein, at least partially drying the harvested plant material, and extracting cannabinoids from the least partially dried plant material, thereby producing an extract comprising cannabinoids.

In another aspect disclosed herein, there is provided an extract derived from the Cannabis plant described herein, or a part thereof, wherein the extract comprises a cannabinoid profile enriched for total CBD, wherein the cannabinoid profile comprises a level of total CBD and a level of total THC at a ratio of from about 10:1 to about 50:1 (CBD:THC), and wherein the level of total CBD is greater than the level of a reference cannabinoid selected from the group consisting of total CBC, total CBG, total CBN, total THCV, and total CBDV.

In another aspect disclosed herein, there is provided a total CBD-enriched cannabinoid extract derived from the Cannabis plant described herein, or a part thereof, wherein the extract comprises total CBD, total THC, and one or more minor cannabinoids selected from the group consisting of: total CBC, total CBG, total CBN, total THCV, total CBDV, total CBL, and total Δ8-THC, wherein the total CBD and the total THC are present in the extract at a ratio of from about 10:1 to about 50:1 (CBD:THC), and wherein the one or more minor cannabinoids is present in the extract in an amount of from about 0.01% to about 10% by weight of the total cannabinoid content of the extract.

In another aspect disclosed herein, there is provided a method for selecting a Cannabis plant comprising a cannabinoid profile enriched for total CBD from a plurality of different Cannabis plants, the method comprising:
(a) harvesting plant material from a plurality of different Cannabis plants;
(b) at least partially drying the harvested plant material of step (a);
(c) measuring in the at least partially dried plant material of step (b) a level of total CBD, total THC and one or more reference cannabinoids selected from the group consisting of THCV, CBDV, CBN, CBC, CBG, THCVA, CBDVA, CBNA, CBCA, and CBGA to generate a cannabinoid profile for each of the plurality of Cannabis plants; and
(d) on the basis of the measurements from step (c), selecting from the plurality of different Cannabis plants a Cannabis plant comprising cannabinoid profile enriched for total CBD and, comprising a level of total CBD and a level of total THC at a ratio of from about 10:1 to about 50:1 (CBD:THC); wherein the total CBD comprises CBD and CBDA, and the total THC comprises THC and THCA; wherein the level of total CBD is greater than the level of a reference cannabinoid selected from the group consisting of:
   (i) total CBC, wherein the total CBC comprises CBC and CBCA;
   (ii) total CBG, wherein the total CBG comprises CBG and CBGA;
   (iii) total CBN, wherein the total CBN comprises CBN and CBNA;
   (iv) total THCV, wherein the total THCV comprises THCV and THCVA; and
   (v) total CBDV, wherein the total CBDV comprises CBDV and CBDVA.

In another aspect disclosed herein, there is provided a method for selecting a Cannabis plant comprising a cannabinoid profile enriched for total CBD from a plurality of different Cannabis plants, the method comprising:
(a) harvesting plant material from a plurality of different Cannabis plants;
(b) at least partially drying the harvested plant material of step (a);
(c) measuring in the at least partially dried plant material of step (b) a level of total CBD, total THC and one or more reference cannabinoids selected from the group consisting of THCV, CBDV, CBN, CBC, CBG, THCVA, CBDVA, CBNA, CBCA, and CBGA to generate a cannabinoid profile for each of the plurality of Cannabis plants; and
(d) measuring in the at least partially dried plant material of step (b) a level of myrcene and a level of β-pinene to generate a terpene profile for each of the plurality of Cannabis plants; and
(e) on the basis of the measurements from step (c) and step (d), selecting from the plurality of different Cannabis plants a Cannabis plant comprising (i) a terpene profile wherein the myrcene is present at a ratio of about 5:1 to the level of β-pinene and (ii) a cannabinoid profile enriched for total CBD and, comprising a level of total CBD and a level of total THC at a ratio of from about 10:1 to about 50:1 (CBD:THC); wherein the total CBD comprises CBD and CBDA, and the total THC comprises THC and THCA; wherein the level of total CBD is greater than the level of a reference cannabinoid selected from the group consisting of:
   (i) total CBC, wherein the total CBC comprises CBC and CBCA;
   (ii) total CBG, wherein the total CBG comprises CBG and CBGA;
   (iii) total CBN, wherein the total CBN comprises CBN and CBNA;
   (iv) total THCV, wherein the total THCV comprises THCV and THCVA; and
   (v) total CBDV, wherein the total CBDV comprises CBDV and CBDVA.

### BRIEF DESCRIPTION OF FIGURES

FIG. 1 shows the relative intensity of (A) CBDA and (B) THCA in Cannabis plants.

FIG. 2 shows the cannabinoid content in a Cannabis plant with a total CBD-enriched cannabinoid profile. (A) A graphical representation of the quantitation of cannabinoid content (y-axis; mg/g) against cannabinoid type (x-axis), inclusive of CBDA. (B) A graphical representation of the quantitation of minor cannabinoid content (y-axis; mg/g) against cannabinoid, exclusive of CBDA.

FIG. 3 shows a graphical representation of the terpene content (y-axis; counts v acquisition time (min)) against relative abundance (x-axis) in a Cannabis plant. (B) A graphical representation of the terpene content (terpene; x-axis) against peak area (counts; y-axis) for Cannabis-1.

FIG. 4 shows the distribution of terpene content in Cannabis plants. (A) Principal component analysis (PCA) of terpene content across Cannabis plants, PCA Scores on PC1 (x-axis; 69.48%) against PCA Scores on PC2 (y-axis; 16.62%). (B) Loadings plot (PC1) demonstrating that myrcene, α-pinene and limonene are in higher abundance (y-axis; 69.48%) against variable (x-axis).

FIG. 5 shows the relative abundance (y-axis; peak area) of (A) β-pinene and (B) myrcene in different Cannabis plants.

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavor to which this specification relates.

Unless specifically defined otherwise, all technical and scientific terms used herein shall be taken to have the same meaning as commonly understood by one of ordinary skill in the art.

Unless otherwise indicated the molecular biology, cell culture, laboratory, plant breeding and selection techniques utilised in the present specification are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present); Janick, J. (2001) Plant Breeding Reviews, John Wiley & Sons, 252 p.; Jensen, N. F. ed. (1988) Plant Breeding Methodology, John Wiley & Sons, 676 p., Richard, A. J. ed. (1990) Plant Breeding Systems, Unwin Hyman, 529 p.; Walter, F. R. ed. (1987) Plant Breeding, Vol. I Theory and Techniques, MacMillan Pub. Co.; Slavko, B. ed. (1990) Principles and Methods of Plant Breeding, Elsevier, 386 p.; and Allard, R. W. ed. (1999) Principles of Plant Breeding, John-Wiley & Sons, 240 p. The ICAC Recorder. Vol. XV no. 2: 3-14; all of which are incorporated by reference. The procedures described are believed to be well known in the art and are provided for the convenience of the reader. All other publications mentioned in this specification are also incorporated by reference in their entirety.

As used in the subject specification, the singular forms “a”, “an” and “the” include plural aspects unless the context clearly dictates otherwise. Thus, for example, reference to “a plant” includes a single plant, as well as two or more plants; reference to “an inflorescence” includes a single inflorescence, as well as two or more inflorescences; and so forth.

As used herein, “and/or” refers to and encompasses any and all possible combinations of one or more of the associated listed items, as well as the lack of combinations when interpreted in the alternative (or).

Amino acid and nucleotide sequences are referred to by sequence identifier numbers (SEQ ID NO), as shown in Table 1, below.

TABLE 1
Amino acid sequence

| SEQ ID | UniProID | | Amino acid sequence |
|---|---|---|---|
| NO: | NO: | Name | |
| 1 | A0A0E3XJ68 | THCA | MNCSAFSFWFVCKIIFFFLSFHIQISIANPRENFLKC |
| | | synthase | FSKHIPNNVANPKLVYTQHDQLYMSILNSTIQNLR |
| | | | FISDTTPKPLVIVTPSNNSHIQATILCSKKVGLQIRT |
| | | | RSGGHDAEGMSYISQVPFVVVDLRNMHSIKIDVH |
| | | | SQTAWVEAGATLGEVYYWINEKNENLSFPGGYC |
| | | | PTVGVGGHFSGGGYGALMRNYGLAADNIIDAHL |
| | | | VNVDGKVLDRKSMGEDLFWAIRGGGGENFGIIA |
| | | | AWKIKLVDVPSKSTIFSVKKNMEIHGLVKLFNKW |
| | | | QNIAYKYDKDLVLMTHFITKNITDNHGKNKTTVH |
| | | | GYFSSIFHGGVDSLVDLMNKSFPELGIKKTDCKEF |
| | | | SWIDTTIFYSGVVNFNTANFKKEILLDRSAGKKTA |
| | | | FSIKLDYVKKPIPETAMVKILEKLYEEDVGAGMY |
| | | | VLYPYGGIMEEISESAIPFPHRAGIMYELWYTASW |
| | | | EKQEDNEKHINWVRSVYNFTTPYVSQNPRLAYLN |
| | | | YRDLDLGKTNHASPNNYTQARIWGEKYFGKNFN |
| | | | RLVKVKTKVDPNNFFRNEQSIPPLPPHHH |

TABLE 2
Single nucleotide polymorphisms (SNPs)

| | | | Refer- | Alter- | |
|---|---|---|---|---|---|
| Variant | PK_Ref | Base | ence | native | SNP |
| No: | Scaffold No. | Pair | Base | Base | Variant |
| 1 | scaffold10076 | 26155 | A | T | A26155T |
| 2 | scaffold10250 | 36989 | A | G | A36989G |
| 3 | scaffold10250 | 39402 | A | G | A39402G |
| 4 | scaffold10250 | 39819 | A | C | A39819C |
| 5 | scaffold10480 | 47002 | A | G | A47002G |
| 6 | scaffold1051 | 29653 | A | C | A29653C |
| 7 | scaffold10903 | 4943 | A | C | A4943C |
| 8 | scaffold11105 | 50059 | G | A | G50059A |
| 9 | scaffold11105 | 54715 | A | T | A54715T |
| 10 | scaffold11691 | 50992 | A | G | A50992G |
| 11 | scaffold11691 | 52937 | T | C | T52937C |
| 12 | scaffold11691 | 52952 | T | A | T52952A |
| 13 | scaffold11691 | 58754 | T | C | T58754C |
| 14 | scaffold11848 | 27836 | A | T | A27836T |
| 15 | scaffold11848 | 52416 | G | T | G52416T |
| 16 | scaffold11848 | 58352 | T | A | T58352A |
| 17 | scaffold11848 | 58399 | C | A | C58399A |
| 18 | scaffold12000 | 17333 | A | T | A17333T |
| 19 | scaffold12051 | 27714 | A | G | A27714G |
| 20 | scaffold1254 | 41485 | G | C | G41485C |
| 21 | scaffold12751 | 37997 | C | T | C37997T |
| 22 | scaffold12943 | 81 | A | C | A81C |
| 23 | scaffold13665 | 5177 | G | T | G5177T |
| 24 | scaffold13665 | 7585 | T | C | T7585C |
| 25 | scaffold13665 | 9184 | A | C | A9184C |
| 26 | scaffold1413 | 12845 | T | C | T12845C |
| 27 | scaffold1413 | 12972 | A | G | A12972G |
| 28 | scaffold14200 | 16627 | A | G | A16627G |
| 29 | scaffold14254 | 36527 | T | G | T36527G |
| 30 | scaffold14254 | 36887 | A | G | A36887G |
| 31 | scaffold14254 | 39282 | A | G | A39282G |
| 32 | scaffold14254 | 44740 | T | C | T44740C |
| 33 | scaffold14254 | 65307 | A | G | A65307G |
| 34 | scaffold14254 | 65347 | A | C | A65347C |
| 35 | scaffold14254 | 78290 | G | A | G78290A |
| 36 | scaffold14528 | 6209 | A | G | A6209G |
| 37 | scaffold15402 | 5227 | C | G | C5227G |
| 38 | scaffold15443 | 6545 | A | C | A6545C |
| 39 | scaffold15546 | 11779 | G | A | G11779A |
| 40 | scaffold16046 | 30110 | A | C | A30110C |
| 41 | scaffold16509 | 3059 | T | C | T3059A |
| 42 | scaffold17281 | 5012 | A | G | A5012G |
| 43 | scaffold182 | 17630 | A | G | A17630G |
| 44 | scaffold18769 | 11158 | C | A | C11158A |
| 45 | scaffold191 | 16236 | C | A | C16236A |
| 46 | scaffold1915 | 26032 | C | A | C26032A |
| 47 | scaffold1915 | 47519 | A | G | A47519G |
| 48 | scaffold19177 | 20011 | T | C | T20011C |
| 49 | scaffold1958 | 15646 | A | G | A15646G |
| 50 | scaffold2087 | 9511 | C | T | C9511T |
| 51 | scaffold2093 | 10893 | A | G | A10893G |
| 52 | scaffold21019 | 11503 | A | T | A11503T |
| 53 | scaffold21320 | 3005 | G | C | G3005C |
| 54 | scaffold21320 | 4087 | T | C | T4087C |
| 55 | scaffold2151 | 4252 | T | G | T4252G |
| 56 | scaffold2151 | 30597 | T | G | T30597G |
| 57 | scaffold2207 | 17476 | A | G | A17476G |
| 58 | scaffold2207 | 17538 | C | T | C17538T |
| 59 | scaffold22813 | 3402 | C | T | C3402T |
| 60 | scaffold22813 | 3516 | A | C | A3516C |
| 61 | scaffold22813 | 3525 | T | C | T3525C |
| 62 | scaffold23157 | 2414 | A | G | A2414G |
| 63 | scaffold23379 | 10166 | A | G | A10166G |
| 64 | scaffold23548 | 15713 | T | A | T15713A |
| 65 | scaffold25092 | 3196 | T | C | T3196C |
| 66 | scaffold25465 | 9356 | G | A | G9356A |
| 67 | scaffold2729 | 16460 | T | C | T16460C |
| 68 | scaffold2729 | 22141 | T | C | T22141C |
| 69 | scaffold2826 | 55472 | C | G | C55472G |
| 70 | scaffold2889 | 146267 | A | G | A146267G |
| 71 | scaffold2889 | 206459 | T | A | T206459A |
| 72 | scaffold29236 | 2022 | A | G | A2022G |
| 73 | scaffold2938 | 21216 | G | A | G21216A |
| 74 | scaffold2938 | 42633 | C | T | C42633T |
| 75 | scaffold2938 | 42666 | T | C | T42666C |
| 76 | scaffold2938 | 42811 | A | G | A42811G |
| 77 | scaffold2997 | 6876 | A | C | A6876C |
| 78 | scaffold2997 | 14778 | T | A | T14778A |
| 79 | scaffold3079 | 35961 | T | C | T35961C |
| 80 | scaffold30901 | 5342 | A | G | A5342G |
| 81 | scaffold31482 | 45021 | T | C | T45021C |
| 82 | scaffold3201 | 7832 | T | C | T7832C |
| 83 | scaffold3201 | 8002 | G | A | G8002A |
| 84 | scaffold3201 | 17293 | A | G | A17293G |
| 85 | scaffold33453 | 1453 | A | C | A1453C |
| 86 | scaffold33453 | 1694 | A | C | A1694C |
| 87 | scaffold33672 | 6476 | T | G | T6476G |
| 88 | scaffold33932 | 892 | A | G | A892G |
| 89 | scaffold36665 | 3583 | A | G | A3583G |
| 90 | scaffold37149 | 36464 | T | A | T36464A |
| 91 | scaffold37149 | 36552 | T | C | T36552C |
| 92 | scaffold37861 | 2586 | A | G | A2586G |
| 93 | scaffold38131 | 13411 | A | G | A13411G |
| 94 | scaffold38131 | 14430 | T | C | T14430C |
| 95 | scaffold40134 | 1797 | A | G | A1797G |
| 96 | scaffold40134 | 1952 | G | C | G1952C |
| 97 | scaffold40134 | 2363 | T | A | T2363A |
| 98 | scaffold40134 | 2771 | G | A | G2771A |
| 99 | scaffold41475 | 1966 | A | G | A1966G |
| 100 | scaffold4156 | 34488 | A | G | A34488G |
| 101 | scaffold43278 | 3709 | G | A | G3709A |
| 102 | scaffold43278 | 3718 | A | G | A3718G |
| 103 | scaffold43278 | 3808 | A | G | A3808G |
| 104 | scaffold43278 | 3815 | T | C | T3815C |
| 105 | scaffold43278 | 4271 | C | T | C4271T |
| 106 | scaffold43278 | 4516 | T | A | T4516A |
| 107 | scaffold43278 | 4545 | G | A | G4545A |
| 108 | scaffold43278 | 4687 | A | G | A4687G |
| 109 | scaffold43278 | 4818 | C | T | C4818T |
| 110 | scaffold44944 | 22434 | T | G | T22434G |
| 111 | scaffold45085 | 285 | T | G | T285G |
| 112 | scaffold45136 | 12235 | A | G | A12235G |
| 113 | scaffold45804 | 29826 | G | A | G29826A |
| 114 | scaffold46506 | 847 | G | A | G847A |
| 115 | scaffold47064 | 1318 | T | G | T1318G |
| 116 | scaffold47064 | 1383 | G | C | G1383C |
| 117 | scaffold47064 | 1398 | T | G | T1398G |
| 118 | scaffold47064 | 1399 | A | G | A1399G |
| 119 | scaffold47064 | 2275 | C | T | C2275T |
| 120 | scaffold47064 | 2459 | C | G | C2459G |
| 121 | scaffold48023 | 16303 | G | A | G16303A |
| 122 | scaffold4808 | 24275 | A | G | A24275G |
| 123 | scaffold4808 | 24441 | A | G | A24441G |
| 124 | scaffold49418 | 949 | C | T | C949T |
| 125 | scaffold49558 | 26979 | A | G | A26979G |
| 126 | scaffold4967 | 70897 | T | C | T70897C |
| 127 | scaffold50427 | 451 | G | C | G451C |
| 128 | scaffold50427 | 487 | A | C | A487C |
| 129 | scaffold50427 | 583 | G | T | G583T |
| 130 | scaffold50427 | 849 | G | A | G849A |
| 131 | scaffold50427 | 857 | G | A | G857A |
| 132 | scaffold50427 | 2148 | T | G | T2148G |
| 133 | scaffold5117 | 11807 | T | C | T11807C |
| 134 | scaffold515 | 17208 | T | C | T17208C |
| 135 | scaffold515 | 17236 | G | A | G17236A |
| 136 | scaffold5374 | 55336 | C | A | C55336A |
| 137 | scaffold55232 | 2067 | G | A | G2067A |
| 138 | scaffold55696 | 4098 | C | G | C4098G |
| 139 | scaffold56241 | 3001 | T | C | T3001C |
| 140 | scaffold56966 | 5347 | A | G | A5347G |
| 141 | scaffold56966 | 6546 | G | A | G6546A |
| 142 | scaffold56966 | 6634 | A | C | A6634C |
| 143 | scaffold56966 | 6699 | A | C | A6699C |
| 144 | scaffold56966 | 6721 | G | T | G6721T |
| 145 | scaffold56966 | 6757 | G | T | G6757T |
| 146 | scaffold57169 | 8417 | G | C | G8417C |
| 147 | scaffold5744 | 2946 | G | T | G2946T |
| 148 | scaffold585 | 49821 | A | G | A49821G |
| 149 | scaffold5876 | 22129 | C | T | C22129T |
| 150 | scaffold5876 | 27987 | G | A | G27987A |
| 151 | scaffold5901 | 12889 | T | G | T12889G |
| 152 | scaffold60476 | 36735 | A | C | A36735C |
| 153 | scaffold6143 | 94084 | G | C | G94084C |
| 154 | scaffold6143 | 96194 | T | C | T96194C |
| 155 | scaffold62752 | 2005 | A | T | A2005T |
| 156 | scaffold63380 | 6980 | T | C | T6980C |
| 157 | scaffold66241 | 4025 | T | A | T4025A |
| 158 | scaffold66363 | 162 | T | C | T162C |
| 159 | scaffold6722 | 110632 | G | A | G110632A |
| 160 | scaffold67269 | 7920 | G | A | G7920A |
| 161 | scaffold67269 | 8040 | C | T | C8040T |
| 162 | scaffold68497 | 462 | T | C | T462C |
| 163 | scaffold68731 | 57121 | A | G | A57121G |
| 164 | scaffold68731 | 58400 | C | T | C58400T |
| 165 | scaffold70404 | 4633 | G | C | G4633C |
| 166 | scaffold70404 | 4661 | G | A | G4661A |
| 167 | scaffold70404 | 4696 | A | C | A4696C |
| 168 | scaffold70404 | 4745 | A | C | A4745C |
| 169 | scaffold7112 | 10919 | G | A | G10919A |
| 170 | scaffold7112 | 14922 | G | A | G14922A |
| 171 | scaffold7112 | 15498 | G | C | G15498C |
| 172 | scaffold7146 | 3687 | T | A | T3687A |
| 173 | scaffold7146 | 3763 | A | C | A3763C |
| 174 | scaffold7146 | 49768 | T | C | T49768C |
| 175 | scaffold7146 | 50455 | A | G | A50455G |
| 176 | scaffold7260 | 12154 | T | C | T12154C |
| 177 | scaffold729 | 29110 | C | G | C29110G |
| 178 | scaffold72919 | 3573 | T | C | T3573C |
| 179 | scaffold72919 | 4236 | G | C | G4236C |
| 180 | scaffold7763 | 3343 | C | G | C3343G |
| 181 | scaffold7763 | 8295 | T | C | T8295C |
| 182 | scaffold7763 | 8839 | T | C | T8839C |
| 183 | scaffold7763 | 19626 | A | G | A19626G |
| 184 | scaffold7763 | 22006 | C | A | C22006A |
| 185 | scaffold9563 | 1657 | A | G | A1657G |
| 186 | scaffold9837 | 9990 | T | A | T9990A |

The present invention is predicated, at least in part, on the inventors' unexpected finding that a Cannabis plant has been generated that comprises an advantageous cannabinoid profile enriched for total CBD (i.e., CBD and CBDA) and further comprises a nucleic acid sequence that encodes a wild-type THCA synthase.

Therefore, in an aspect disclosed herein, there is provided a Cannabis plant, or a part thereof, comprising a cannabinoid profile enriched for total CBD, wherein the cannabinoid profile comprises a level of total CBD and a level of total THC at a ratio of from about 10:1 to about 50:1 (CBD:THC); wherein the total CBD comprises cannabidiol (CBD) and cannabidiolic acid (CBDA), and the total THC comprises Δ-9-tetrahydrocannabinol (THC) and Δ-9-tetrahydrocannabinolic acid (THCA); wherein the level of total CBD is greater than the level of a reference cannabinoid selected from the group consisting of:
(a) total CBC, wherein the total CBC comprises cannabichromene (CBC) and cannabichromene acid (CBCA);
(b) total CBG, wherein the total CBG comprises cannabigerol (CBG) and cannabigerolic acid (CBGA);
(c) total CBN, wherein the total CBN comprises cannabinol (CBN) and cannabinolic acid (CBNA);
(d) total THCV, wherein the total THCV comprises tetrahydrocannabivarin (THCV) and tetrahydrocannabivarinic acid (THCVA); and
(e) total CBDV, wherein the total CBDV comprises cannabidivarin (CBDV) and cannabidivarinic acid (CBDVA),
   and wherein the Cannabis plant comprises a nucleic acid sequence that encodes a wild-type THCA synthase.

### Cannabis

As used herein, the term “Cannabis plant” means a plant of the genus Cannabis, illustrative examples of which include Cannabis sativa, Cannabis indica and Cannabis ruderalis. Cannabis is an erect annual herb with a dioecious breeding system, although monoecious plants exist. Wild and cultivated forms of Cannabis are morphologically variable, which has resulted in difficulty defining the taxonomic organisation of the genus. In an embodiment, the Cannabis plant is C. sativa.

The terms “plant”, “cultivar”, “variety”, “strain” or “race” are used interchangeably herein to refer to a plant or a group of similar plants according to their structural features and performance (i.e., morphological and physiological characteristics).

The reference genome for C. sativa is the assembled draft genome and transcriptome of “Purple Kush” or “PK” (van Bakal et al. 2011, Genome Biology, 12: R102). C. sativa, has a diploid genome (2n=20) with a karyotype comprising nine autosomes and a pair of sex chromosomes (X and Y). Female plants are homogametic (XX) and males heterogametic (XY) with sex determination controlled by an X-to-autosome balance system. The estimated size of the haploid genome is 818 Mb for female plants and 843 Mb for male plants.

As used herein, the term “part” refers to any part of the plant, illustrative examples of which include an embryo, a shoot, a bud, a root, a stem, a seed, a stipule, a leaf, a petal, an inflorescence, an ovule, a bract, a trichome, a branch, a petiole, an internode, bark, a pubescence, a tiller, a rhizome, a frond, a blade, pollen and stamen. The term “part” also includes any material listed in the Plant Part Code Table as approved by the Australian Therapeutic Goods Administration (TGA) Business Services (TBS). In an embodiment, the part is selected from the group consisting of an embryo, a shoot, a bud, a root, a stem, a seed, a stipule, a leaf, a petal, an inflorescence, an ovule, a bract, a trichome, a branch, a petiole, an internode, bark, a pubescence, a tiller, a rhizome, a frond, a blade, pollen and stamen. In a preferred embodiment, the part is a Cannabis bud.

### Cannabinoids

The term “cannabinoid”, as used herein, refers to a family of terpeno-phenolic compounds, of which more than 100 compounds are known to exist in nature. Cannabinoids will be known to persons skilled in the art, illustrative examples of which are provided in Table 3, below, including acidic and decarboxylated forms thereof.

TABLE 3
Cannabinoids and their properties

| | | Chemical |
|---|---|---|
| | | properties/ |
| Name | Structure | [M + H]⁺ ESI MS |
| Δ9- tetrahydrocannabinol (THC) | 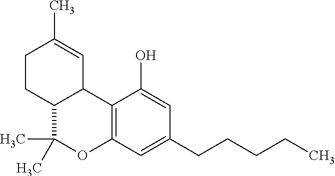 | Psychoactive, decarboxylation product of THCA m/z 315.2319 |
| Δ9- tetrahydrocannabinolic acid (THCA) | 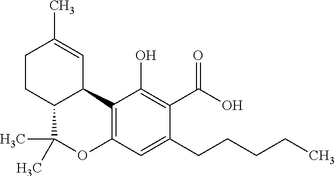 [SMILES: CCCCCc1cc2c(c(O)c1C(=O)O)[C@@H]1C=C(C)CC[C@H]1C(C)(C)O2] | m/z 359.2217 |
| cannabidiol (CBD) | 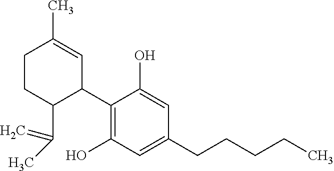 [SMILES: C=C(C)C1CCC(C)=CC1c1c(O)cc(CCCCC)cc1O] | decarboxylation product of CBDA m/z 315.2319 |
| cannabidiolic acid (CBDA) | 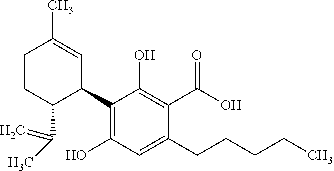 [SMILES: C=C(C)[C@@H]1CCC(C)=C[C@H]1c1c(O)cc(CCCCC)c(C(=O)O)c1O] | m/z 359.2217 |
| cannabigerol (CBG) | 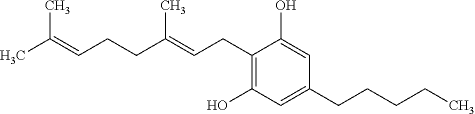 [SMILES: CCCCCc1cc(O)c(C/C=C(\C)CCC=C(C)C)c(O)c1] | Non-intoxicating, decarboxylation product of CBGA m/z 317.2475 |
| cannabigerolic acid (CBGA) | 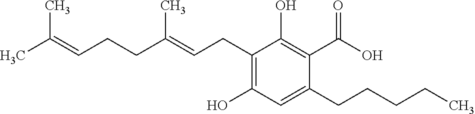 [SMILES: CCCCCc1cc(O)c(C/C=C(\C)CCC=C(C)C)c(O)c1C(=O)O] | m/z 361.2373 |
| cannabichromene (CBC) | 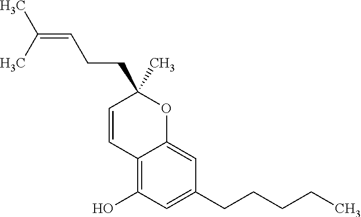 [SMILES: CCCCCc1cc(O)c2c(c1)O[C@](C)(CCC=C(C)C)C=C2] | Non- psychotropic, converts to cannabicyclol upon light exposure m/z 315.2319 |
| cannabichromene acid (CBCA) | 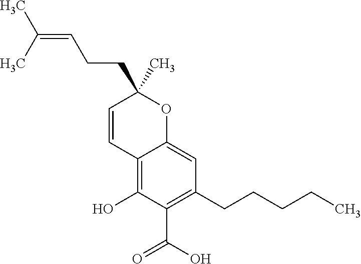 [SMILES: CCCCCc1cc2c(c(O)c1C(=O)O)C=C[C@@](C)(CCC=C(C)C)O2] | m/z 359.2217 |
| cannabicyclol (CBL) | 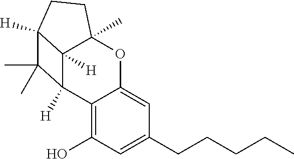 | Non- psychoactive, 16 isomers known. Derived from non-enzymatic conversion of CBC m/z 315.2319 |
| cannabinol (CBN) | 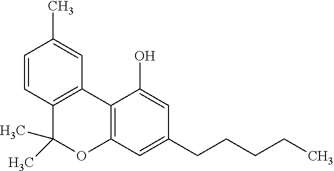 [SMILES: CCCCCc1cc(O)c2c(c1)OC(C)(C)c1ccc(C)cc1-2] | Likely degradation product of THC m/z 311.2006 |
| cannabinolic acid (CBNA) | 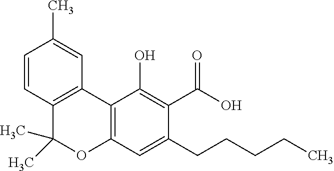 [SMILES: CCCCCc1cc2c(c(O)c1C(=O)O)-c1cc(C)ccc1C(C)(C)O2] | m/z 355.1904 |
| tetrahydrocannabivarin (THCV) | 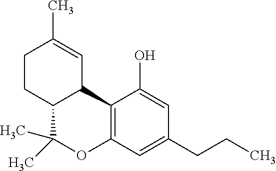 | decarboxylation product of THCVA m/z 287.2006 |
| tetrahydrocannabivarinic acid (THCVA) | 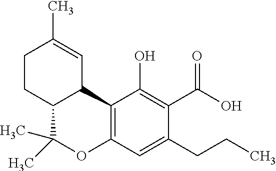 | m/z 331.1904 |
| cannabidivarin (CBDV) | 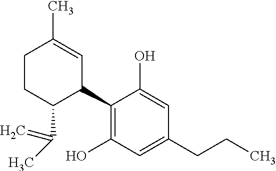 | m/z 287.2006 |
| cannabidivarinic acid (CBDVA) | 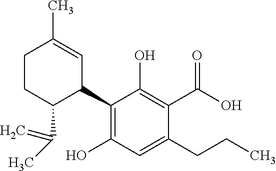 | m/z 331.1904 |
| Δ8-tetrahydrocannabinol (d8-THC) | 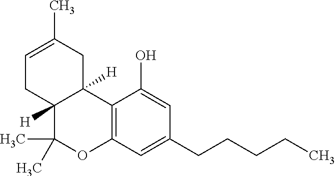 [SMILES: CCCCCc1cc(O)c2c(c1)OC(C)(C)[C@@H]1CC=C(C)C[C@@H]21] | m/z 315.2319 |

Cannabinoids are synthesised in Cannabis plants as carboxylic acids. While some decarboxylation may occur in the plant, decarboxylation typically occurs post-harvest and is increased by exposing plant material to heat (Sanchez and Verpoote, 2008, Plant Cell Physiology, 49(12): 1767-82). Decarboxylation is usually achieved by drying and/or heating the plant material. Persons skilled in the art would be familiar with methods by which decarboxylation of cannabinoids can be promoted, illustrative examples of which include air-drying, combustion, vaporisation, curing, heating and baking.

### Cannabinoid Profile

The term “cannabinoid profile” refers to a representation of the type, amount, level, ratio and/or proportion of cannabinoids that are present in the Cannabis plant or part thereof, as typically measured within plant material derived from the plant or part, including an extract therefrom.

The term “enriched” is used herein to refer to a selectively higher level of one or more cannabinoids in the Cannabis plant or part thereof. For example, a cannabinoid profile enriched for total CBD refers to plant material in which the amount of total CBD (total CBD and total CBDA) is greater than the amount of any of the other cannabinoids that may also be present (including constitutively present) in the plant material.

The cannabinoid profile in a Cannabis plant will typically predominantly comprise the acidic form of the cannabinoids, but may also comprise some decarboxylated (neutral) forms thereof, at various concentrations or levels at any given time (i.e., at propagation, growth, harvest, drying, curing, etc). Thus, the term “total cannabinoid” is used herein to refer to the decarboxylated and the acid form of said cannabinoid. For example, “total CBD” refers to CBD and CBDA, “total THC” refers to THC and THCA, “total CBC” refers to CBC and CBCA, “total CBG” refers to CBG and CBGA, “total CBN” refers to CBN and CBNA, “total THCV” refers to THCV and THCVA, “total CBDV” refers to CBDV and CBDVA, and so forth.

The terms “level”, “content”, “concentration” and the like, are used interchangeably herein to describe an amount of the referenced compound, and may be represented in absolute terms (e.g., mg/g, mg/ml, etc.) or in relative terms, such as a ratio to any or all of the other compounds in the Cannabis plant material or as a percentage of the amount (e.g., by weight, peak area, etc.) of any or all of the other compounds in the Cannabis plant material.

As used herein, the term “plant material” is to be understood to mean any part of the Cannabis plant, including the leaves, stems, roots, and inflorescence, or parts thereof, as described elsewhere herein, as well as extracts, illustrative examples of which include kief or hash, which includes trichomes and glands. In an embodiment, the plant material is female inflorescence.

The term “inflorescence” as used herein means the complete flower head of the Cannabis plant, comprising stems, stalks, bracts, flowers and trichomes (i.e., glandular, sessile and stalked trichomes).

“Cannabidiolic acid” or “CBDA” is a derivative of cannabigerolic acid (CBGA), which is converted to CBDA by CBDA synthase. Its neutral form, “cannabidiol” or “CBD” has antagonist activity on agonists of the CB1 and CB2 receptors. CBD has also been shown to act as an antagonist of the putative cannabinoid receptor, GPR55. CBD is commonly associated with therapeutic or medicinal effects of Cannabis and has been suggested for use as a sedative, anti-inflammatory, anti-anxiety, anti-nausea, atypical anti-psychotic, and as a cancer treatment. CBD can also increase alertness, and attenuate the memory impairing effect of THC.

The Cannabis plant described herein comprises a cannabinoid profile that is characterised by a level of total CBD in the plant material that is greater than the level of total THC. Accordingly, the Cannabis plant of the invention may be variously described as “high-CBD”, “CBD-enriched” or “high-CBD, low-THC”. Those skilled in the art would understand this terminology to mean a Cannabis plant that produced higher levels of CBD and CBDA relative to the level of THC and THCA.

In an embodiment, the level of total CBD is at least about 80% by weight of the total cannabinoid content of the dry weight of plant material, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material.

“Δ-9-tetrahydrocannabinolic acid” or “THCA” is also synthesised from the CBGA precursor by THCA synthase. The neutral form “Δ-9-tetrahydrocannabinol” is associated with psychoactive effects of Cannabis, which are primarily mediated by its activation of CB1G-protein coupled receptors, which result in a decrease in the concentration of cyclic AMP (cAMP) through the inhibition of adenylate cyclase. THC also exhibits partial agonist activity at the cannabinoid receptors CB1 and CB2. CB1 is mainly associated with the central nervous system, while CB2 is expressed predominantly in the cells of the immune system. As a result, THC is also associated with pain relief, relaxation, fatigue, appetite stimulation, and alteration of the visual, auditory and olfactory senses. Furthermore, more recent studies have indicated that THC mediates an anti-cholinesterase action, which may suggest its use for the treatment of Alzheimer's disease and myasthenia (Eubanks et al., 2006, Molecular Pharmaceuticals, 3(6): 773-7).

In an embodiment, the level of total THC is from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of plant material.

In an embodiment, the level of total CBD and the level of total THC are present at a ratio of from about 10:1 to about 50:1, preferably from about 10:1 to about 40:1, preferably from about 10:1 to about 30:1, preferably from about 15:1 to about 50:1, preferably from about 15:1 to about 40:1, preferably from about 15:1 to about 30:1, preferably from about 20:1 to about 50:1, preferably from about 20:1 to about 40:1, or more preferably from about 20:1 to about 30:1 (CBD:THC).

The reference cannabinoids disclosed herein may be alternatively described as “minor cannabinoids” or “secondary cannabinoids”.

Minor cannabinoids have been shown to exhibit unique medicinal properties. For example, CBDV has been given orphan designation by the European Medicines Agency for use in the treatment of Rhett Syndrome and Fragile X Syndrome (EU/3/17/1921). THCV has also been recognised as new potential treatment against obesity-associated glucose intolerance (Wargent et al., 2013, Nutrition & Diabetes, 3: e68). The therapeutic applications of other minor cannabinoids, such as CBC, CBG and CBN have also been reviewed by, for example, Izzo et al. (2009, Trends in Pharmacological Sciences, 30(10): 515-527) and Morabito et al. (2013, Current Addiction Reports, 3(2): 230-238).

In an embodiment, the reference cannabinoid is total CBC. In another embodiment, the level of total CBD is present at a ratio of from about from about 10:1 to about 50:1 to the level of total CBC, preferably from about 10:1 to about 40:1, preferably from about 10:1 to about 30:1, preferably from about 15:1 to about 50:1, preferably from about 15:1 to about 40:1, preferably from about 15:1 to about 30:1, preferably from about 20:1 to about 50:1, preferably from about 20:1 to about 40:1, or more preferably from about 20:1 to about 30:1 (CBD:CBC).

In another embodiment, the level of total CBC is from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material.

In an embodiment, the reference cannabinoid is total CBG. In another embodiment, the level of total CBD is present at a ratio of from about from about 10:1 to about 100:1 to the level of total CBG, preferably from about 10:1 to about 90:1, preferably from about 10:1 to about 80:1, preferably from about 20:1 to about 100:1, preferably from about 20:1 to about 90:1, preferably from about 20:1 to about 80:1, preferably from about 30:1 to about 100:1, preferably from about 30:1 to about 90:1, preferably from about 30:1 to about 80:1, preferably from about 40:1 to about 100:1, preferably from about 40:1 to about 90:1, preferably from about 40:1 to about 80:1, preferably from about 50:1 to about 100:1, preferably from about 50:1 to about 90:1, preferably from about 50:1 to about 80:1, preferably from about 60:1 to about 100:1, preferably from about 60:1 to about 90:1, preferably from about 60:1 to about 80:1, preferably from about 70:1 to about 100:1, preferably from about 70:1 to about 90:1, or more preferably from about 70:1 to about 80:1 (CBD:CBG).

In another embodiment, the level of total CBG is from about 1% and 5%, preferably from about 1% and 4%, preferably from about 1% and 3%, or more preferably from about 1% and 2% by weight of the total cannabinoid content of the dry weight of plant material.

In an embodiment, the reference cannabinoid is total CBN. In another embodiment, the level of total CBD is present at a ratio of from about from about 2000:1 to about 3000:1 of the level of total CBN, preferably from about 2000:1 to about 3000:1, preferably from about 2100:1 to about 3000:1, preferably from about 2200:1 to about 3000:1, preferably from about 2300:1 to about 3000:1, preferably from about 2400:1 to about 3000:1, or more preferably from about 2500:1 to about 3000:1 (CBD:CBN).

In another embodiment, the level of total CBN is from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of plant material.

In an embodiment, the reference cannabinoid is total CBDV. In another embodiment, the level of total CBD is present at a ratio of is from about 10:1 to about 80:1 to the level of total CBDV, preferably from about 10:1 to about 70:1, preferably from about 20:1 to about 80:1, preferably from about 20:1 to about 70:1, preferably from about 30:1 to about 80:1, preferably from about 30:1 to about 70:1, preferably from about 40:1 to about 80:1, preferably from about 40:1 to about 70:1, preferably from about 50:1 to about 80:1, preferably from about 50:1 to about 70:1, preferably from about 60:1 to about 80:1, or more preferably from about 60:1 to about 70:1 (CBD:CBDV).

In another embodiment, the level of total CBDV in the plant material is from about 1% to about 10%, preferably from about 1% to about 9%, preferably from about 1% to about 8%, preferably from about 1% to about 7%, preferably from about 1% to about 6%, or more preferably from about 1% to about 5% by weight of the total cannabinoid content of the of dry weight of plant material.

In an embodiment, the reference cannabinoid is total THCV. In another embodiment, the level of total CBD is present at a ratio of from about 400:1 to about 700:1 of the level of total THCV, preferably from about 400:1 to about 600:1, preferably from about 500:1 to about 700:1, or more preferably from about 500:1 to about 600:1 (CBD:THCV).

In another embodiment, the level of total THCV is from about 0.05% to about 1%, preferably from about 0.05% to about 0.09%, preferably from about 0.05% to about 0.08%, preferably from about 0.05% to about 0.07%, preferably from about 0.05% to about 0.06%, preferably from about 0.05% to about 0.04%, preferably from about 0.05% to about 0.03%, or more preferably from about 0.05% to about 0.02% by weight of the total cannabinoid content of the dry weight of plant material.

In an embodiment, the Cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material; and
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of plant material;
(iii) optionally a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material;
(iv) optionally a level of total CBG of from about 1% and 5%, preferably from about 1% and 4%, preferably from about 1% and 3%, or more preferably from about 1% and 2% by weight of the total cannabinoid content of the dry weight of plant material;
(v) optionally a level of total CBN of from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of plant material;
(vi) optionally a level of total CBDV of from about 1% to about 10%, preferably from about 1% to about 9%, preferably from about 1% to about 8%, preferably from about 1% to about 7%, preferably from about 1% to about 6%, or more preferably from about 1% to about 5% by weight of the total cannabinoid content of the of dry weight of plant material; and
(vii) optionally a level of total THCV of from about 0.05% to about 1%, preferably from about 0.05% to about 0.09%, preferably from about 0.05% to about 0.08%, preferably from about 0.05% to about 0.07%, preferably from about 0.05% to about 0.06%, preferably from about 0.05% to about 0.04%, preferably from about 0.05% to about 0.03%, or more preferably from about 0.05% to about 0.02% by weight of the total cannabinoid content of the dry weight of plant material.

In an embodiment, the Cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material; and
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of plant material.

In an embodiment, the Cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material;
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of plant material; and
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material.

In an embodiment, the Cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material;
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material; and
(iv) a level of total CBG of from about 1% and 5%, preferably from about 1% and 4%, preferably from about 1% and 3%, or more preferably from about 1% and 2% by weight of the total cannabinoid content of the dry weight of plant material.

In an embodiment, the Cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material;
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material;
(iv) a level of total CBG of from about 1% and 5%, preferably from about 1% and 4%, preferably from about 1% and 3%, or more preferably from about 1% and 2% by weight of the total cannabinoid content of the dry weight of the plant material; and
(v) a level of total CBN of from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of plant material.

In an embodiment, the Cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material;
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material;
(iv) a level of total CBG of from about 1% and 5%, preferably from about 1% and 4%, preferably from about 1% and 3%, or more preferably from about 1% and 2% by weight of the total cannabinoid content of the dry weight of plant material;
(v) a level of total CBN of from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of plant material; and
(vi) a level of total CBDV of from about 1% to about 10%, preferably from about 1% to about 9%, preferably from about 1% to about 8%, preferably from about 1% to about 7%, preferably from about 1% to about 6%, or more preferably from about 1% to about 5% by weight of the total cannabinoid content of the of dry weight of plant material.

In an embodiment, the Cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material;
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material;
(iv) a level of total CBG of from about 1% and 5%, preferably from about 1% and 4%, preferably from about 1% and 3%, or more preferably from about 1% and 2% by weight of the total cannabinoid content of the dry weight of the plant material;
(v) a level of total CBN of from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of plant material;
(vi) a level of total CBDV of from about 1% to about 10%, preferably from about 1% to about 9%, preferably from about 1% to about 8%, preferably from about 1% to about 7%, preferably from about 1% to about 6%, or more preferably from about 1% to about 5% by weight of the total cannabinoid content of the of dry weight of plant material; and
(vii) a level of total THCV of from about 0.05% to about 1%, preferably from about 0.05% to about 0.09%, preferably from about 0.05% to about 0.08%, preferably from about 0.05% to about 0.07%, preferably from about 0.05% to about 0.06%, preferably from about 0.05% to about 0.04%, preferably from about 0.05% to about 0.03%, or more preferably from about 0.05% to about 0.02% by weight of the total cannabinoid content of the dry weight of plant material.

In an embodiment, the Cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material; and/or
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of plant material; and/or
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material; and/or
(iv) a level of total CBG of from about 1% and 5%, preferably from about 1% and 4%, preferably from about 1% and 3%, or more preferably from about 1% and 2% by weight of the total cannabinoid content of the dry weight of plant material; and/or
(v) a level of total CBN of from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of plant material; and/or
(vi) a level of total CBDV of from about 1% to about 10%, preferably from about 1% to about 9%, preferably from about 1% to about 8%, preferably from about 1% to about 7%, preferably from about 1% to about 6%, or more preferably from about 1% to about 5% by weight of the total cannabinoid content of the of dry weight of plant material; and/or
(vii) a level of total THCV of from about 0.05% to about 1%, preferably from about 0.05% to about 0.09%, preferably from about 0.05% to about 0.08%, preferably from about 0.05% to about 0.07%, preferably from about 0.05% to about 0.06%, preferably from about 0.05% to about 0.04%, preferably from about 0.05% to about 0.03%, or more preferably from about 0.05% to about 0.02% by weight of the total cannabinoid content of the dry weight of plant material.

In another aspect, there is provided a seed of the Cannabis plants described herein. As used herein, “seed” refers to immature seeds which are developing in planta. According to another aspect disclosed herein, there is provided a progeny plant, or a part thereof, which is produced from the seed.

### Terpenes

The term “terpene” as used herein, refers to a class of organic hydrocarbon compounds, which are produced by a variety of plants. Cannabis plants produce and accumulate different terpenes, such as monoterpenes and sesquiterpenes, in the glandular trichomes of the female inflorescence. The term “terpene” includes “terpenoids” or “isoprenoids”, which are modified terpenes that contain additional functional groups.

Terpenes are responsible for much of the scent of Cannabis flowers and contribute to the unique flavor qualities of Cannabis products. Terpenes will be known to persons skilled in the art, illustrative examples of which are provided in Table 4.

TABLE 4
Terpenes and their properties

| | | Mass/Charge number |
|---|---|---|
| Name | Structure | (m/z)* |
| α-Phellandrene | 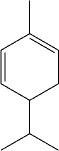 | m/z 93.0 |
| α-Pinene (+/−) | 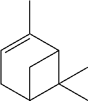 | m/z 93.0 |
| Camphene | 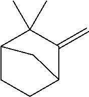 | m/z 93.0 |
| β-Pinene (+/−) | 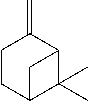 | m/z 93.0 |
| Myrcene | 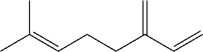 | m/z 93.0 |
| Limonene | 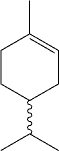 | m/z 68.1 |
| 3-Carene | 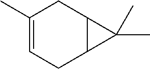 | |
| Eucalyptol | 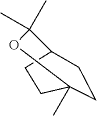 | m/z 81.0 |
| γ-Terpinene | 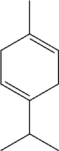 | m/z 93.1 |
| Linalool | 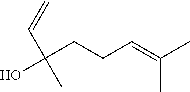 | m/z 93.0 |
| γ-Elemene | 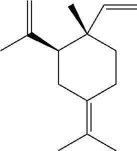 | m/z 121.0 |
| Humulene | 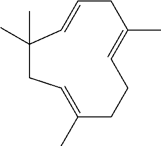 | m/z 93.0 |
| Nerolidol | 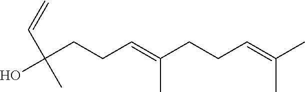 [SMILES: C=CC(C)(O)CC/C=C(\C)CCC=C(C)C] | m/z 222.4 |
| Guaia-3,9-diene | 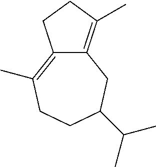 | m/z 161.1 |
| Caryophyllene | 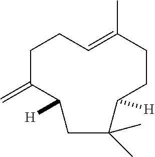 | m/z 69.2 |

*The molecular ion is not necessarily seen for all compounds

Terpene biosynthesis in plants typically involves two pathways to produce the general 5-carbon isoprenoid diphosphate precursors of all terpenes: the plastidial methylerythritol phosphate (MEP) pathway and the cytosolic mevalonate (MEV) pathway. These pathways control the different substrate pools available for terpene synthases (TPS).

Terpenes have been shown to exhibit unique medicinal properties as described, for example, by Brahmkshatriya and Brahmkshatriya (2013, in Ramawat and Mérillon (eds), Natural Products, Springer, Berlin, Heidelberg).

### Terpene Profile

The term “terpene profile” as used herein refers to a representation of the type, amount, level, ratio and/or proportion of terpenes that are present in the Cannabis plant or part thereof, as typically measured within plant material derived from the plant or plant part, including an extract therefrom.

The terpene profile in a Cannabis plant will be determined based on genetic, environmental and developmental factors, therefore particular terpenes may be present at various amounts, levels, ratios and/or proportions at any given time (i.e., at propagation, growth, harvest, drying, curing, etc).

In an embodiment, the terpene profile comprises monoterpenes and sesquiterpenes.

Monoterpenes consist of two isoprene units and may be liner or contain ring structures. The primary function of monoterpenes is to protect plants from infection by fungal and bacterial pathogens and insect pests. Monoterpenes would be known to persons skilled in the art, illustrative embodiments of which include α-phellandrene, α-pinene, camphene, β-pinene, myrcene, limonene, eucalyptol, γ-terpinene and linalool.

Sesquiterpenes differ from other common terpenes as they contain one additional isoprene unit, which creates a 15 carbon structure. The primary function of sesquiterpenes is as a pheromone for the bud and flower. Sesquiterpenes would be known to persons skilled in the art, illustrative embodiments of which include γ-elemene, humulene, nerolidol, guaia-3,9-diene and caryophyllene.

In an embodiment, the terpene profile comprises a level of sesquiterpenes that correlates with the level of total CBD. In a preferred embodiment, the terpene profile comprises a high level of sesquiterpenes that correlates with a high level of total CBD.

In an embodiment, the terpene profile in the Cannabis plant comprises terpenes selected from the group consisting of α-phellandrene, α-pinene, camphene, β-pinene, myrcene, limonene, eucalyptol, γ-terpinene, linalool, γ-elemene, humulene, nerolidol, guaia-3,9-diene and caryophyllene.

In a preferred embodiment, the terpene profile in the Cannabis plant comprises terpenes selected from the group consisting of myrcene and β-pinene.

“Myrcene” is a monoterpinoid derivative of β-pinene. Myrcene has been associated with the therapeutic or medicinal effects of Cannabis and has been suggested for use as a sedative, hypnotic, analgesic and muscle relaxant. Myrcene is also hypothesised to attenuate the activity of other cannabinoids and terpenes as part of the “entourage effect” as described in, for example, Russo, 2011, British Journal of Pharmacology, 163(7): 1344-1364.

“β-pinene” is a monoterpene that is characterised by a woody-green, pine-like scent. β-pinene has been shown to act as a topical antiseptic and a bronchodilator. β-pinene is also capable of crossing the blood-brain barrier and it is hypothesised that β-pinene inhibits the influence of THC as part of the entourage effect, as described elsewhere herein.

In an embodiment, the level of myrcene is present in a ratio of about 5:1 to the level of β-pinene.

### Nucleic Acids

As noted elsewhere herein, the inventors have surprisingly found that the Cannabis plant described herein comprises a CBD-enriched cannabinoid profile and a nucleic acid sequence that encodes a wild-type THCA synthase. This is unexpected because CBD and THC are derived from the same precursor molecule, CBGA, which is processed to THCA via THCA synthase. Hence, the presence of a wild-type THCA synthase should otherwise have favoured the synthesis of THCA and produced a higher amount of THCA in the plant material of the Cannabis plants described herein.

Preferably, the wild-type THCA synthase comprises the amino acid sequence shown in SEQ ID NO: 1 or an amino acid sequence having at least 85% sequence identity thereto. By “at least 85%” means at least 85%, preferably at least 86%, preferably at least 87%, preferably at least 88%, preferably at least 89%, preferably at least 90%, preferably at least 91%, preferably at least 92%, preferably at least 93%, preferably 94%, preferably at least 95%, preferably at least 96%, preferably at least 97%, preferably at least 98%, or more preferably at least 99%.

The Cannabis plant disclosed herein may be further defined by reference to its genotype. The characteristic genotype includes, but is not limited to, a single nucleotide polymorphism (SNP) profile that is associated with a total CBD-enriched cannabinoid profile.

The terms “single nucleotide polymorphism” and “SNP” as used herein to refer to a variation to a single nucleotide at a specific position in the genome, where each variation is present to some appreciable degree within the population comprising the genome.

In an embodiment, the Cannabis plant comprises one or more SNP(s) selected from the group consisting of Variant Nos: 1-186, as shown in Table 2 herein.

The terms “polynucleotide”, “polynucleotide sequence”, “nucleotide sequence”, “nucleic acid” or “nucleic acid sequence” as used interchangeably herein to designate mRNA, RNA, cRNA, cDNA or DNA. The term typically refers to polymeric form of nucleotides of at least 10 bases in length, either ribonucleotides or deoxynucleotides or a modified form or either type of nucleotide. The term includes single and double stranded forms of RNA and DNA.

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

In another aspect disclosed herein, there is provided a seed of the Cannabis plants described herein. As used herein, “seed” refers to immature seeds which are developing in planta. According to another aspect disclosed herein, there is provided a Cannabis plant, or a part thereof, which is produced from the seed.

### Tissue Culture

In another aspect disclosed herein, there is provided a tissue culture of regenerable cells derived from the Cannabis plant described herein, or a part thereof. In another aspect, there is provided a Cannabis plant generated from the tissue culture, wherein the plant expresses the morphological and physiological characteristics of the Cannabis plant described herein.

As used herein, the phrase “tissue culture” refers to a population of cells or protoplasts, including plant calli and plant tissue clumps, derived from the Cannabis plant described herein that are maintained in vitro and from which a further Cannabis plant can be generated.

Suitable techniques for establishing a tissue culture and regenerating plants therefrom will be well known to persons skilled in the art, illustrative examples of which are described in Vasil (1984), Cell Culture and Somatic Cell Genetics of Plants, Vol I, II, III Laboratory Procedures and Their Applications, Academic Press, New York; Green et al. (1987), Plant Tissue and Cell Culture, Academic Press, New York; Weissbach and Weissbach (1989), Methods for Plant Molecular Biology, Academic Press; Gelvin et al. (1990), Plant Molecular Biology Manual, Kluwer Academic Publishers; and Evans et al. (1983) Handbook of Plant Cell Culture, MacMillian Publishing Company, New York.

In an embodiment, the tissue culture comprises a population of cells or protoplast of a plant part selected from the group consisting of seeds, leaves, stems, pollen, anthers, ovules, embryos, preferably cotyledons or hypocotyls. In a preferred embodiment, the population of cells or protoplast are from the scutellum of immature embryos, mature embryo, callus derived therefrom, or meristematic tissue.

### Breeding Techniques

In another aspect, there is provided a method for producing an F1 hybrid Cannabis plant, the method comprising crossing the Cannabis plant, as described herein, with a different Cannabis plant to produce an F1 hybrid.

By way of example, the Cannabis plant described herein, is manually crossed with other Cannabis plants. The resulting “Filial generation 1” or “F1” plants are self-fertilised and the resulting F2 generation plants, which will typically show large variability on account of gene segregation, are planted in a selection field. These F2 plants are observed during the growing season for phenotypic traits such as health, growth, vigour, plant type, plant structure, leaf type, flowering, maturity and inflorescent yield. F2 plants with the desirable trait(s) are selected, harvested, and the female inflorescent analysed for cannabinoid profile. The seeds of the selected F2 plants can be cleaned and stored. This procedure may be repeated, whereby the selection and testing units increase from individual plants in the F2, to multiple plants containing ‘lines’ (descending from one mother plant) in the F5 and the number of units decrease from approximately 500 plants in the F2 to 20 lines in the F5 by selecting about 10-20% of the units in each selection cycle. The increased size of the units, whereby more seed per unit is available, allows the selection and testing in replicated trials on more than one location with a different environment and a more extensive and accurate analysing of the cannabinoid profile. The lines or candidate varieties become genotypically more homozygous and phenotypically more homogeneous by selecting similar plant types within a line and by discarding the so-called off-types from the very variable F2 generation on to the final F7 or F8 generation. Depending on the intermediate results, the plant breeder may decide to vary the procedure such as by accelerating the process by testing a particular line earlier or retesting a line. They may also select plants for further crossing with existing parent plants or with other plants resulting from the current selection procedure.

The Cannabis plant and parts thereof, as herein described, including F1 and subsequent generations derived therefrom, may be further exposed to mutagenesis and/or marker assisted selection, as is known to persons skilled in the art, to generate and/or select for new plants with desirable phenotypic, chemotypic and/or genotypic profiles. This can provide non-transgenic Cannabis plants that are free of exogenous nucleic acid molecule, thereby avoiding the restrictions that otherwise apply to genetically-modified organisms (GMO), including plants, in some countries/regions. Typically, a progenitor plant cell, tissue, seed or plant is exposed to mutagenesis to produce single or multiple point mutations, such as nucleotide substitutions, deletions, additions and/or codon modification.

Methods for performing mutagenesis on plants or plant parts will be familiar to persons skilled in the art, illustrative examples of which include chemical or radiation-induced mutagenesis, for example EMS or sodium azide treatment of seed, or gamma irradiation. Chemical mutagenesis typically favours nucleotide substitutions rather than deletions. Heavy ion beam (HIB) irradiation is known as an effective technique for mutation breeding to produce new plant cultivars. Ion beam irradiation has two physical factors, the dose (gy) and LET (linear energy transfer, keV/um) for biological effects that determine the amount of DNA damage and the size of DNA deletion, and these can be adjusted according to the desired extent of mutagenesis.

Biological agents suitable for site-directed mutagenesis include enzymes that include double stranded breaks in DNA that stimulate endogenous repair mechanisms. Illustrative examples include endonucleases, zinc finger nucleases (ZFNs), TAL effector nuclease (TALENs), transposases and site-specific recombinases. ZFNs, for example, facilitate site-specific cleavage within a genome allowing endogenous or other end-joining repair mechanisms to introduce deletions or insertions to repair the gap.

Isolation of mutants may be achieved by screening mutagenised plants or seed. For example, a mutagenised population of wheat may be screened directly for the desired genotype or indirectly by screening for a phenotype (i.e., cannabinoid profile). Screening directly for the genotype preferably includes assaying for the presence of mutations which may be observed in PCR assays by the absence of markers as expected when some of the genes are deleted, or heteroduplex based assays, or by deep sequencing. Screening for the phenotype may comprise quantitative analysis of cannabinoids, as provided by the Examples. Using this methodology, large populations of mutagenised Cannabis strains may be screened for a desired cannabinoid profile.

Identified mutations may then be introduced into desirable genetic backgrounds by crossing the mutant with a plant of the desired genetic background and performing a suitable number of backcrosses to cross out the originally undesired parent background.

An “induced” or “introduced” mutation is to be understood to mean an artificially induced genetic variation that may be the result of chemical or radiation treatment of a progenitor seed or plant. Nucleotide insertional derivatives include 5′ and 3′ terminal fusions as well as intra-sequence insertions of single or multiple nucleotides. Insertional nucleotide sequence variants are those in which one or more nucleotides are introduced into a site in the nucleotide sequence, either at a predetermined site as is possible with ZFNs, TALENs or homologous recombination methods, or by random insertion with suitable screening of the resulting product. Deletional variants are typically characterised by the removal of one or more nucleotides from the sequence. A mutant gene may have only a single insertion of a sequence of nucleotides relative to the wild-type gene and one or more substitution mutations. Substitutional nucleotide variants are typically those in which at least one nucleotide in the sequence has been removed and a different nucleotide inserted in its place. Preferably, the number of nucleotides affected by substitutions in a mutant gene relative to the wild-type gene is no more than 10, preferably no more than 9, preferably no more than 8, preferably no more than 7, preferably no more than 6, preferably no more than 5, preferably no more than 4, preferably no more than 3, preferably no more than 2, preferably no more than 1 nucleotide.

The term “mutation”, as used herein, will typically not include a silent nucleotide substitution; that is, a mutation that does not affect the activity of the gene, and therefore includes only alterations in the gene sequence which affects the gene activity. The term “polymorphism” refers to any change in the nucleotide sequence including such silent nucleotide substitutions. Screening methods may first involve screening for polymorphisms and secondly for mutations within a group of polymorphic variants.

Marker-assisted selection is a well-recognised method of selecting for heterozygous plants required when backcrossing with a recurrent parent in a classical breeding program. The population of plants in each backcross generation will be heterozygous for the gene of interest normally present in a 1:1 ratio in a backcross population, and the molecular marker can be used to distinguish the two alleles of the gene. By extracting DNA from, for example, young shoots and testing with a specific marker for the introgressed desirable trait, early selection of plants for further backcrossing is made whilst energy and resources are concentrated on fewer plants. To further speed up the backcrossing program, the embryo from immature seeds (25 days post anthesis) may be excised and grown up on nutrient media under sterile conditions, rather than allowing full seed maturity.

### Transgenic Plants

In another aspect, there is provided a method for producing a transgenic Cannabis plant, the method comprising transfecting the Cannabis plant described herein, or a part thereof, with a heterologous nucleic acid sequence. In another aspect, there is provided a transgenic Cannabis plant produced by the methods disclosed herein, or a seed or progeny plant derived therefrom.

In an embodiment, the transduced heterologous nucleic acid sequence introduces one or more nucleic acid substitutions, deletions, or additions into the genome of the Cannabis plant.

Nucleic acid constructs useful for producing the above-mentioned transgenic plants can readily be produced using standard techniques known to persons skilled in the art. To ensure appropriate expression of the gene encoding an mRNA of interest, the nucleic acid construct typically comprises one or more regulatory elements such as promoters, enhancers, as well as transcription termination or polyadenylation sequences. Such elements are well known in the art. The transcriptional initiation region comprising the regulatory element(s) may provide for regulated or constitutive expression in the plant. The regulatory elements may be selected from, for example, seed-specific promoters, or promoters not specific for seed cells (such as ubiquitin promoter or CaMV35S or enhanced 35S promoters). The promoter may be modulated by factors such as temperature, light or stress. Ordinarily, the regulatory elements will be provided 5′ of the genetic sequence to be expressed. The construct may also contain other elements that enhance transcription such as the nos 3′ or the ocs 3′ polyadenylation regions or transcription terminators.

Typically, the nucleic acid construct comprises a selectable marker. Selectable markers aid in the identification and screening of plants or cells that have been transformed with the exogenous nucleic acid molecule. The selectable marker gene may provide antibiotic or herbicide resistance to the Cannabis cells, or allow the utilisation of substrates such as mannose.

Preferably, the nucleic acid construct is stably incorporated into the genome of the plant. Accordingly, the nucleic acid comprises appropriate elements which allow the molecule to be incorporated into the genome, or the construct is placed in an appropriate vector which can be incorporated into a chromosome of a plant cell.

The terms “transgenic plant” and “transgenic Cannabis plant”, as used herein, typically refer to a plant that contains a gene construct (“transgene”) not found in a wild-type plant of the same species, variety or cultivar. That is, transgenic plants (transformed plants) contain genetic material that they did not contain prior to the transformation. A “transgene” as referred to herein has the normal meaning in the art of biotechnology and refers to a genetic sequence which has been produced or altered by recombinant DNA or RNA technology and which has been introduced into a progenitor plant cell, which cell is used to produce a new plant. The transgene may include genetic sequences obtained from or derived from a plant cell, or another plant cell, or a non-plant source, or a synthetic sequence. Typically, the transgene has been introduced into the plant by human manipulation such as, for example, by transformation but any method can be used as one of skill in the art recognizes. The genetic material is typically stably integrated into the genome of the plant. The introduced genetic material may comprise sequences that naturally occur in the same species but in a rearranged order or in a different arrangement of elements, for example an antisense sequence or a sequence encoding a double-stranded RNA or an artificial microRNA precursor. Plants containing such sequences are included herein in “transgenic plants”. Transgenic plants as defined herein include all progeny of an initial transformed and regenerated plant (TO plant) which has been genetically modified using recombinant techniques, where the progeny comprise the transgene. Such progeny may be obtained by self-fertilisation of the primary transgenic plant or by crossing such plants with another plant of the same species. In an embodiment, the transgenic plant comprises the introduction of one of more nucleic acid substitutions, deletions or additions into the genome of the Cannabis plant of the invention. In another embodiment, the transgenic plants are homozygous for each and every gene that has been introduced (transgene) so that their progeny do not segregate for the desired phenotype. Transgenic plant parts include all parts and cells of said plants which comprise the transgene such as, for example, seeds, cultured tissues, callus and protoplasts. A “non-transgenic plant”, preferably a non-transgenic Cannabis plant, is one which has not been genetically modified by the introduction of genetic material by recombinant DNA techniques.

In an embodiment, the transgenic plants are produced by transfecting the Cannabis plant of the invention with a heterologous nucleic acid sequence.

Transformation of a nucleic acid molecule into a cell can be accomplished by any method by which a nucleic acid molecule can be inserted into the cell. Illustrative examples of suitable transformation techniques include transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained. Preferred host cells are plant cells, more preferably cells of a Cannabis plant.

Any of several methods may be employed to determine the presence of a transgene in a transformed plant, as are known to persons skilled in the art. By way of example, polymerase chain reaction (PCR) may be used to amplify sequences that are unique to the transformed plant, with detection of the amplified products by gel electrophoresis or other methods. DNA may be extracted from the plants using conventional methods and the PCR reaction carried out using primers that will distinguish the transformed and non-transformed plants. An alternative method to confirm a positive transformant is by Southern blot hybridisation, well known in the art. Cannabis plants which are transformed may also be identified (i.e. distinguished from non-transformed or wild-type Cannabis plants) by their phenotype, the presence of a selectable marker gene, by immunoassays that detect or quantify the expression of an enzyme encoded by the transgene, or any other phenotype conferred by the transgene.

Transgenic plants, as described herein, include plants and their progeny which have been genetically modified using recombinant techniques. This would generally be to modulate the production of at least one polypeptide defined herein in the desired plant or plant organ. Transgenic plant parts include all parts and cells of said plants such as, for example, cultured tissues, callus and protoplasts. Transformed plants contain genetic material that they did not contain prior to the transformation. The genetic material is preferably stably integrated into the genome of the plant. The introduced genetic material may comprise sequences that naturally occur in the same species but in a rearranged order or in a different arrangement of elements, for example an antisense sequence. Such plants are included herein as “transgenic plants”. A “non-transgenic plant” is one which has not been genetically modified with the introduction of genetic material by recombinant DNA techniques. In a preferred embodiment, the transgenic plants are homozygous for each and every gene that has been introduced (transgene) so that their progeny do not segregate for the desired phenotype.

### Cannabis Extracts

In another aspect, there is provided a method of producing an extract comprising cannabinoids from a Cannabis plant, the method comprising the steps of:
(a) harvesting plant material from the Cannabis plant described herein;
(b) at least partly drying the harvested plant material of (a); and
(c) extracting cannabinoids from the at least partly dried plant material of (b), thereby producing an extract comprising cannabinoids.

In an embodiment, the extract comprises a cannabinoid profile enriched for total CBD, wherein the cannabinoid profile comprises a level of total CBD and a level of total THC at a ratio of from about 10:1 and 50:1 (CBD:THC), and wherein the level of total CBD is greater than the level of a reference cannabinoid selected from the group consisting of total CBC, total CBG, total CBN, total THCV, and total CBDV.

In another embodiment, the extract of cannabinoids comprises total CBD, total THC, and one or more minor cannabinoids selected from the group consisting of total CBC, total CBG, total CBN, total THCV, total CBDV, and total Δ8-THC, wherein the total CBD and the total THC are present in the extract at a ratio of from about 10:1 and 50:1 (CBD:THC), and wherein the one or more minor cannabinoids is present in the extract in an amount from about 0.01% to about 10% by weight of the total cannabinoid content of the solution.

The term “extract”, as used herein, is to be understood as including a whole Cannabis extract, such as resin, hash and keif, as well as substantially purified compounds isolated from the harvested plant material, such as cannabinoids, terpenes and/or flavonoids.

As used herein, “substantially purified” refers to a compound or molecule that has been isolated from other components with which it is typically associated in its native state (i.e., within the plant material). Preferably, the substantially purified molecule is at least 60% free, more preferably at least 75% free, and more preferably at least 90% free from other components with which it is naturally associated. By “isolated” is meant material that is substantially or essentially free from components that normally accompany it in its native state.

Persons skilled in the art would recognise that isolated cannabinoids may exists as a number of different chemical species, illustrative examples of which include salts, solvates, prodrugs, stereoisomers or tautmers thereof.

The term “drying” as used herein refers to any method for drying the plant material. Illustrative examples include air-drying, curing, and heat drying. In an embodiment, the plant material is dried in a temperature, light and humidity controlled environment, such as a temperature of about 21° C. and a humidity of from about 38% and 45% RH. In another embodiment, heat is applied to the plant material during the drying process to cure the dried plant material. Temperatures suitable for curing dried plant material would be known to persons skilled in the art, illustrative examples of which include a temperature from about 60° C. to about 225° C., preferably from about 100° C. to about 150° C., preferably from about 110° C. to about 130° C., or more preferably about 120° C. In an embodiment, the dried plant material is cured by heating the dried plant material at about 120° C. for 2 hours.

It is to be understood that the terms “dry”, “drying” and the like are not intended to mean the absence of moisture in the plant material, and therefore includes any state in which at least some moisture has been removed from the plant material. Persons skilled in the art will be familiar with the extent to which Cannabis plant material can be dried to allow for extraction of the desirable compound(s), including decarboxylated cannabinoids. In an embodiment, the harvested plant material is dried under conditions and for a period of time that gives rise to a loss of at least 5%, preferably at least 10%, preferably at least 20%, preferably at least 30%, preferably at least 40%, preferably at least 50%, preferably at least 60%, preferably at least 70%, preferably at least 80%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% of the moisture content of the plant material at the time of harvest.

Methods of extracting cannabinoids from plant material would be known to persons skilled in the art, illustrative examples of which include supercritical fluid extraction (SFE). The principles of SFE relate to the disappearance of the gas-liquid boundary when the temperature of certain materials was increased by heating them in a closed glass container. This allows the material to reach its critical point, which is the temperature above which a substance or compound can co-exist in the gas, liquid and solid phases. By taking substances to their critical point and at pressure, SFE can be used as sophisticated solvents for extraction and fractionation of complex mixtures. SFE is commonly used in the processing of oil and has also been applied to the purification and separation of vegetable and fish oils. More recently SFE has been used to extract cannabinoids from plant material, for example, method for the extraction of pharmaceutically active cannabinoids from plant material is provided in WO/2004/016277, the contents of which is incorporated herein by reference.

In an embodiment, cannabinoids are extracted from the dried plant material by SFE.

In an embodiment, the plant material comprises female inflorescence.

In another aspect disclosed herein, there is provided an extract produced by the methods described herein.

The present disclosure provides an extract derived from the Cannabis plant described herein, or a part thereof, comprising a cannabinoid profile enriched for total CBD, wherein the cannabinoid profile comprises a level of total CBD and a level of total THC at a ratio of from about 10:1 and 50:1 (CBD:THC), and wherein the level of total CBD is greater than the level of a reference cannabinoid selected from the group consisting of total CBC, total CBG, total CBN, total THCV, and total CBDV.

The present disclosure also provides an extract derived from the Cannabis plant described herein, or a part thereof, comprising CBD, THC, and one or more minor cannabinoids selected from the group consisting of: CBC, CBG, CBN, THCV, CBDV, CBL, and Δ8-THC, wherein the total CBD and the total THC are present in the extract at a ratio of from about 10:1 to about 50:1 (CBD:THC); and wherein the one or more minor cannabinoids is present in the extract in an amount of from about 0.01% to about 10% by weight of the total cannabinoid content of the extract.

### Methods for Selecting Cannabis Plants

The present disclosure enables the identification and selection of Cannabis plants with a particular beneficial cannabinoid profile (i.e. a cannabinoid profile enriched for total CBD).

In an embodiment, the selected Cannabis plants, or parts thereof, can be used for medical purpose. In another embodiment, the selected Cannabis plants, or parts thereof, can be used in the treatment, or for the amelioration of symptoms associated with, a disease. Suitable diseases will be known to persons skilled in the art, illustrative examples of which include acquired hypothyroidism, acute gastritis, agoraphobia, AIDS-related illness, alcohol abuse, alcoholism, alopecia areata, Alzheimer's Disease, amphetamine dependency, amyloidosis, amyotrophic lateral sclerosis (ALS), angina pectoris, ankylosis, anorexia, anorexia nervosa, anxiety disorders, any chronic medical symptom that limits major life activities, arteriosclerotic heart disease, arthritis, arthropathy, gout, asthma, attention deficit hyperactivity disorder (ADD/ADHD), Autism/Asperger's, autoimmune disease, back pain, back sprain, Bell's Palsy, bipolar disorder, bruxism, bulimia, cachexia, cancer, carpal tunnel syndrome, cerebral palsy, cervical disk disease, cervicobrachial syndrome, chronic fatigue, syndrome, chronic pain, chronic renal failure, cocaine dependence, colitis, conjunctivitis, constipation, Crohn's Disease, cystic fibrosis, Darier's Disease, delirium tremens, dermatomyositis, diabetes, diabetic neuropathy, diabetic peripheral vascular disease, diarrhea, diverticulitis, dysthymic disorder, eczema, emphysema, endometriosis, epidermolysis bullosa, epididymitis, epilepsy, Felty's Syndrome, fibromyalgia, Friedreich's Ataxia, gastritis, genital herpes, Graves' Disease, headaches, Hemophilia A, Henoch-Schonlein Purpura, Hepatitis C, hereditary spinal ataxia, HIV/AIDS, Huntington's Disease, hypertension, hyperventilation, hypoglycemia, impotence, inflammatory autoimmune-mediated arthritis, inflammatory bowel disease (IBD), insomnia, intermittent explosive disorder (TIED), Lou Gehrig's Disease, Lyme Disease, melorheostosis, Meniere's Disease, motion sickness, mucopolysaccharidosis (MPS), Multiple Sclerosis (MS), muscle spasms, muscular dystrophy, Nail-Patella Syndrome, nightmares, obesity, obsessive compulsive disorder, opiate dependence, osteoarthritis, panic disorder, Parkinson's Disease, peripheral neuropathy, pain, persistent insomnia, porphyria, Post-Polio Syndrome (PPS), Post-Traumatic Stress Disorder (PTSD), premenstrual syndrome (PMS), prostatitis, psoriasis, pulmonary fibrosis, Raynaud's Disease, Reiter's Syndrome, Restless Legs Syndrome (RLS), rosacea, schizoaffective disorder, schizophrenia, scoliosis, sedative dependence, seizures, senile dementia, severe nausea, shingles (Herpes Zoster), sinusitis, skeletal muscular spasticity, sleep apnoea, sleep disorders, spasticity, spinal stenosis, Sturge-Weber Syndrome (SWS), stuttering, Tardive Dyskinesia (TD), temporomandibular joint disorder (TMJ), tenosynovitis, thyroiditis, Tietze's Syndrome, tinnitus, tobacco dependence, Tourette's Syndrome, trichotillomania, viral hepatitis, wasting syndrome, Wittmaack-Ekbom's Syndrome, nausea, and vomiting.

Accordingly, in another aspect disclosed herein, there is provided a method for selecting a Cannabis plant comprising a cannabinoid profile enriched for total CBD from a plurality of different Cannabis plants, the method comprising:
(a) harvesting plant material from a plurality of different Cannabis plants;
(b) at least partially drying the harvested plant material of step (a);
(c) measuring in the at least partially dried plant material of step (b) a level of total CBD, total THC and one or more reference cannabinoids selected from the group consisting of THCV, CBDV, CBN, CBC, CBG, THCVA, CBDVA, CBNA, CBCA, and CBGA to generate a cannabinoid profile for each of the plurality of Cannabis plants; and
(d) on the basis of the measurements from step (c), selecting from the plurality of different Cannabis plants a Cannabis plant comprising cannabinoid profile enriched for total CBD and, comprising a level of total CBD and a level of total THC at a ratio of from about 10:1 to about 50:1 (CBD:THC), wherein the total CBD comprises CBD and CBDA, and the total THC comprises THC and THCA, wherein the level of total CBD is greater than the level of a reference cannabinoid selected from the group consisting of:
   (i) total CBC, wherein the total CBC comprises CBC and CBCA;
   (ii) total CBG, wherein the total CBG comprises CBG and CBGA;
   (iii) total CBN, wherein the total CBN comprises CBN and CBNA;
   (iv) total THCV, wherein the total THCV comprises THCV and THCVA; and
   (v) total CBDV, wherein the total CBDV comprises CBDV and CBDVA.

The terms “selecting” or “selection” as used herein means the selection of one or more Cannabis plants from the plurality of different Cannabis plants based on the cannabinoid profile of the individual Cannabis plant. The term “plurality” is to be understood to mean more than 1 (e.g., 2, 3, 4, 5, 6, 7, 8, 9, 10, 11, etc.).

In an embodiment, the method further comprises
(a) measuring in the at least partially dried plant material of step (b) a level of myrcene and a level of β-pinene to generate a terpene profile for each of the plurality of Cannabis plants; and
(b) on the basis of the measurements from step (e), selecting from the plurality of different Cannabis plants a Cannabis plant comprising terpene profile wherein the myrcene is present in a ratio of about 5:1 to the level of β-pinene.

Thus, in another aspect disclosed herein, there is provided a method for selecting a Cannabis plant comprising a cannabinoid profile enriched for total CBD from a plurality of different Cannabis plants, the method comprising:
(a) harvesting plant material from a plurality of different Cannabis plants;
(b) at least partially drying the harvested plant material of step (a);
(c) measuring in the at least partially dried plant material of step (b) a level of total CBD, total THC and one or more reference cannabinoids selected from the group consisting of THCV, CBDV, CBN, CBC, CBG, THCVA, CBDVA, CBNA, CBCA, and CBGA to generate a cannabinoid profile for each of the plurality of Cannabis plants; and
(d) measuring in the at least partially dried plant material of step (b) a level of myrcene and a level of β-pinene to generate a terpene profile for each of the plurality of Cannabis plants; and
(e) on the basis of the measurements from step (c) and step (d), selecting from the plurality of different Cannabis plants a Cannabis plant comprising (i) a terpene profile wherein the myrcene is present in a ratio of about 5:1 to the level of β-pinene and (ii) a cannabinoid profile enriched for total CBD and, comprising a level of total CBD and a level of total THC at a ratio of from about 10:1 to about 50:1 (CBD:THC), wherein the total CBD comprises CBD and CBDA, and the total THC comprises THC and THCA, wherein the level of total CBD is greater than the level of a reference cannabinoid selected from the group consisting of:
   (i) total CBC, wherein the total CBC comprises CBC and CBCA;
   (ii) total CBG, wherein the total CBG comprises CBG and CBGA;
   (iii) total CBN, wherein the total CBN comprises CBN and CBNA;
   (iv) total THCV, wherein the total THCV comprises THCV and THCVA; and
   (v) total CBDV, wherein the total CBDV comprises CBDV and CBDVA.

In an embodiment, the selected Cannabis plant is crossed with a different Cannabis plant to produce a F1 hybrid.

In an embodiment, regenerable cells isolated from the selected Cannabis plant are transformed with a heterologous nucleic acid sequence and cultured for a time and under conditions suitable to produce a transgenic Cannabis plant.

In an embodiment, regenerable cells isolated from the selected Cannabis plant are transfected with a gene editing construct comprising a nucleic acid sequence encoding a DNA-recognition moiety and cultured for a time and under conditions suitable to produce a non-transgenic Cannabis plant with modified gene expression.

Persons skilled in the art would understand that the DNA-recognition moiety may be DNA, RNA or a polypeptide.

Illustrative examples of suitable DNA molecules include antisense, as well as sense (e.g., coding and/or regulatory) DNA molecules. Antisense DNA molecules include short oligonucleotides. Other examples of inhibitory DNA molecules include those encoding interfering RNAs, such as shRNA and siRNA. Yet another illustrative example of an inhibitor of gene expression is catalytic DNA, also referred to as DNAzymes.

Illustrative examples of suitable RNA molecules include siRNA, dsRNA, stRNA, shRNA and miRNA (e.g. short temporal RNAs and small modulatory RNAs), ribozymes, and guide (i.e., gRNA or single-guide RNA (sgRNA)) or clustered regularly interspaced short palindromic repeats (CRISPR) RNAs used in combination with the Cas or other endonucleases (van der Oost et al. 2014, Nature Reviews Microbiology, 12(7):479-92).

In an embodiment the DNA-recognition moiety is a CRISPR RNA. Suitable CRISPR RNA will be known to persons skilled in the art, illustrative examples of which include guide RNA (gRNA) and single-guide RNA (sgRNA).

In an embodiment the DNA-recognition moiety is a polypeptide. Illustrative examples of a suitable polypeptide molecules are “Zinc finger nucleases” or “ZFN”, as described elsewhere herein.

The terms “guide RNA” or “gRNA” refer to a RNA sequence that is complementary to a target DNA and directs a CRISPR endonuclease to the target DNA. gRNA comprises crispr RNA (crRNA) and a tracr RNA (tracrRNA). crRNA is a 17-20 nucleotide sequence that is complementary to the target DNA, while the tracrRNA provides a binding scaffold for the endonuclease. crRNA and tracrRNA exist in nature a two separate RNA molecules, which has been adapted for molecular biology techniques using, for example, 2-piece gRNAs such as CRISPR tracer RNAs (cr:tracrRNAs).

The terms “single-guide RNA” or “sgRNA” refers to a single RNA sequence that comprises the crRNA fused to the tracrRNA.

Accordingly, the skilled person would understand that the term “gRNA” describes all CRISPR guide formats, including two separate RNA molecules or a single RNA molecule. By contrast, the term “sgRNA” will be understood to refer to single RNA molecules combining the crRNA and tracrRNA elements into a single nucleotide sequence.

In a preferred embodiment, the DNA-recognition moiety is a single-guide RNA (sgRNA).

In an embodiment, the targeting gene editing construct further comprises a nucleic acid encoding an endonuclease.

Suitable endonucleases will be known to persons skilled in the art, illustrative examples of which include an RNA-guided DNA endonuclease, zinc finger nuclease (ZFN), transcription activator-like effector nucleases (TALEN), CRISPR-associated (Cas) nucleases.

In an embodiment, the nuclease is selected from the group consisting of an RNA-guided DNA endonuclease, ZFN, and a TALEN.

“Transcription activator-like effector nucleases” or “TALEN” are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind practically any desired DNA sequence, so when combined with a nuclease, DNA can be cut at specific locations. The restriction enzymes can be introduced into cells, for use in gene editing or for genome editing in situ, a technique known as genome editing with engineered nucleases. The mechanism of TALEN-mediated cleavage of target DNA sequences would be known to persons skilled in the art and has been described, for example by Boch (2011, Nature Biotechnology, 29: 135-136), Juong et al. (2013, Nature Reviews Molecular Cell Biology, 14: 49-55) and Sune et al. (2013, Biotechnology and Bioengineering, 110: 1811-1821).

“Zinc finger nucleases” or “ZFN” are proteins comprising nucleic acid binding domains that are stabilised by zinc. The individual DNA binding domains are typically referred to as “fingers”, such that a ZFN has at least one finger, preferably two fingers, preferably three fingers, preferably four fingers, preferably five fingers, or more preferably six fingers. Each finger binds from two to four base pairs of a target DNA sequence, and typically comprises an about 30 amino acid zinc-chelating, DNA binding region. ZFN facilitate site-specific cleavage within a target DNA sequence, allowing endogenous or other end-joining repair mechanisms to introduce insertions or deletions to repair the gap. The mechanism of ZFN-mediated cleavage of target DNA sequences would be known to persons skilled in the art and has been described, for example, by Liu et al. (2010, Biotechnology and Bioengineering, 106: 97-105).

In an embodiment, the RNA-guided DNA endonuclease is a CRISPR-associated (Cas) endonuclease.

The CRISPR-Cas system evolved in bacteria and archaea as an adaptive immune system to defend against viral attack. Upon exposure to a virus, short segments of viral DNA are integrated in the clustered regularly interspaced short palindromic repeats (i.e., CRISPR) locus. RNA is transcribed from a portion of the CRISPR locus that includes the viral sequence. That RNA, which contains sequence complementarity to the viral genome, mediates targeting of a Cas endonuclease to the sequence in the viral genome. The Cas endonuclease cleaves the viral target sequence to prevent integration or expression of the viral sequence.

The mechanisms of CRISPR-mediated gene editing would be known to persons skilled in the art and have been described, for example, by Doudna et al., (2014, Methods in Enzymology, 546) and Belhaj et al., (2013, Plant Methods, 9:39) and in WO 2013/188638 and WO 2014/093622.

Suitable Cas endonucleases will be known to persons skilled in the art, illustrative examples of which include Cas9, Cas12a (also referred to as Cpf1), Cas12b (also referred to as C2c1), Cas13a (also referred to as C2c2), Cas13b, CasX, Cas3 and Cas10. The term “Cas endonucleases” as used herein also contemplates the use of natural and engineered Cas endonucleases, described, for example, by Wu et al. (2018, Nature Chemical Biology, 14: 642-651).

In a preferred embodiment, the Cas endonuclease is Cas9.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

The various embodiments enabled herein are further described by the following non-limiting examples.

### EXAMPLES

### A. Materials

### Plants

Cannabis plants were grown under an Office of Drug Control licence at the Victorian Government Medicinal Cannabis Cultivation Facility, Victoria, Australia. Indoor greenhouse growing facilities were equipped with full climate control (i.e., temperature, humidity and high-intensity lighting) to ensure that crops were produced in almost identical growing conditions.

Cannabis plants were asexually propagated from cuttings taken from vegetative mother plants originating from a single seed source. Cuttings were maintained for 2 weeks at 22° C. in a high humidity environment (i.e., 50% relative humidity) under 18 hours day light in rooting medium to stimulate root development before being transferred to substrate medium for hydroponic growth. The plants were grown for a further 5 weeks under the same growth conditions before being transferred to a larger substrate medium to induce flowering.

Flowering conditions were identical to the rooting and growth conditions, with the exception that the day light length was reduced to 12 hours. The plants were maintained in flowing conditions for 9 weeks to allow for flowering and maturation.

The plants were irrigated throughout their growing cycle with potable quality water and sustained release fertilizer is applied to the soil-free medium.

Upon maturation, plants were harvested at the base of the plant and dried in a temperature and humidity controlled environment (i.e., approximately 21° C. at approximately 38-45% humidity) for between 3 to 5 weeks prior to extraction or analysis, as described below.

### Reagents and Standards

All HPLC grade reagents, water with 0.1% formic acid (mobile phase A), acetonitrile with 0.1% formic acid (mobile phase B) and methanol were obtained from Fisher Scientific (Fair Lawn, N.J.). Primary standards for CBDA and THCA in acetonitrile, and CBD, CBN, CBC, THC in methanol, at 1000 μg/mL, were commercially purchased from Novachem Pty Ltd (Heidelberg West, Australia) as distributor for Cerilliant Corporation (Round Rock, Tex.). A mixed stock standard at 125 μg/mL CBDA, CBN, CBC, THCA and 250 μg/mL CBD, THC in methanol was prepared with working standards at 0.05, 0.125, 0.25, 0.5, 1.25, 2.5 and 50.0 μg/mL for CBDA, CBN, CBC and THCA; and 0.1, 0.25, 0.5, 1.0, 2.5, 5.0 and 100.0 μg/mL for CBD and THC prepared from the mixed stock. Primary standards for THCV, CBDV, CBG, THCVA, CBNA, CBCA, CBGA, CBL and Δ8-THC in methanol, at 1000 μg/mL, were commercially purchased from Novachem Pty Ltd (Heidelberg West, Australia) as distributor for Cerilliant Corporation (Round Rock, Tex.). These were combined to make a 100 μg/mL stock (i.e. 100 uL taken and mixed from each). This mixed standard was diluted to 0.1, 0.25, 0.5, 1.0, 2.5, 5.0 and 100 μg/mL. All standards were stored at −80° C.

### B. Sample Preparation

Inflorescences were separated from the plant material from 69 different female Cannabis cultivars. Samples were ground to a fine powder with liquid nitrogen using a SPEX SamplePrep 2010 Geno/Grinder for 1 minute at 1500 rpm. After grinding, 10 mg of each sample was weighed into an Axygen 2.0 mL microcentrifuge tube on a Sartorius BP210D analytical balance. Each sample was extracted with 1 mL of methanol, vortexed for 30 seconds, sonicated for 5 minutes and centrifuged at 13,000 rpm for 5 minutes. The supernatant was transferred to a 2 mL amber HPLC vial and diluted 1:3 for analysis.

Where necessary, plant material was cured by heating the ground dried plant material at 120° C. for 2 hours.

### C. Liquid Chromatography/Mass Spectroscopy Analysis

Samples were analysed using a Thermo Scientific (Waltham, Mass.) Q Exactive Plus Orbitrap mass spectrometer (MS) coupled with Thermo Scientific Vanquish ultra-high performance liquid chromatography (UHPLC) system equipped with degasser, binary pump, temperature controlled autosampler and column compartment, and photodiode array detector (PDA).

Separation was carried out using a C18 column (Phenomenex Luna Omega, 1.6 μm, 150 mm×2.1 mm) maintained at 30° C. with water and acetonitrile (both with 0.1% formic acid) as mobile phases and a flow rate of 0.3 mL/min. The separation gradient is described in Table 5.

The MS was set to acquire a full range spectrum (80-1,200 m/z) followed by a data independent MS2 spectrum in positive polarity with resolution set to 35,000. The capillary temperature was set to 320° C. with sheath and auxiliary gas at 28 and 15 units respectively and a spray voltage of 4 kV. PDA data acquisition was set to a data collection rate of 5 Hz from 190 and 680 nm.

TABLE 5
Separation gradient for LCMS analysis

| Time | % A | % B |
|---|---|---|
| (min) | (Water with 0.1% FA) | (Acetonitrile with 0.1% FA) |
| 0 | 60.0 | 40.0 |
| 2.0 | 60.0 | 40.0 |
| 3.0 | 25.0 | 75.0 |
| 10.0 | 10.0 | 90.0 |
| 11.0 | 0.0 | 100.0 |
| 15.0 | 0.0 | 100.0 |
| 15.1 | 60.0 | 40.0 |
| 20.0 | 60.0 | 40.0 |

### D. Static Headspace Solid Phase Micro-Extraction, Liquid Extraction and Gas Chromatography/Mass Spectroscopy Analysis

Terpenes were extracted from 20 mg of milled, dried Cannabis biomass using static headspace with direct injection, headspace solid phase micro-extraction (SPME) or liquid extraction using hexane followed by chromatographic separation on an Agilent 7000 GC-QQQ using a DB-5, DB-17 or VF-35 capillary GC column. The optimal column for separation of the volatiles was the DB-5 column. Static headspace was effective for the analysis of extract monoterpenes, while sesquiterpenes were more effectively extracted using a hexane-based liquid extraction method. Final analytical conditions for the static headspace analysis are provided in Table 6 and final conditions for the liquid extraction are presented in Table 7.

TABLE 6
HS and GC-MC parameters used for relative
determination of terpenes from cannabis
GC-MS parameters for static headspace analysis

| | |
|---|---|
| Sample | 20 mg milled, dried cannabis bud |
| Incubation (pre- | 5 min |
| extraction) | |
| Extraction | 30 min |
| Desorption | 2 min |
| Fibre post-bake | 30 min |

GC-MS Parameters

| Column | DB-5, DB-17, VF-35 | |
|---|---|---|
| Oven | Time (min) | Temperature (° C.) |
| | 0 | 60 |
| | 2 | 60 |
| | 7 | 110 |
| | 12 | 110 |
| | 13.5 | 125 |
| | 18.5 | 125 |
| | 26 | 200 |
| | 28 | 300 |
| | 31 | 300 |
| | TOTAL RUN TIME | 31 min |

| | |
|---|---|
| Carrier gas | Helium, Flow: 1.6221 mL/min at 1.5 psi |
| Injector | 10:1 split, 250° C., injection volume 1000 μL |
| Headspace | Incubation temperature 130° C. for 5 min, |
| | injection volume 1000 μL |
| Scan range | 29-350 |

TABLE 7
Liquid extraction and GC-MS parameters used for
quantitative determination of terpenes from cannabis
GC-MS parameters for liquid injection analysis

| Sample | 20 mg milled, dried cannabis bud, extracted twice | |
|---|---|---|
| | with 200 μL hexane. | |
| | Extracts combined for analysis | |
| Oven | Time (min) | Temperature (° C.) |
| | 0 | 60 |
| | 2 | 60 |
| | 30 | 200 |
| | 31 | 320 |
| | 34 | 320 |
| | TOTAL RUN TIME | 35 min |

| | |
|---|---|
| Carrier gas | Helium, Flow: 1.6221 mL/min at 1.5 psi |
| Injector | 1:5 split, 250° C., 1 μL |
| Flow rates and | Main Run |
| Backflush | Column 1: flow 1.3801 mL/min; average velocity |
| settings | 32.251 cm/sec; |
| | Column 2: 0.50013 psi, flow 1.428 mL/min; average |
| | velocity 151.6 cm/sec |
| | Backflush |
| | Column 1: 3 min (post-run) - 1.9815 mL/min; |
| | Column 2: 3 min (post-run) - 7.0194 mL/min. |

### E. Data Processing

### Chemometric Analysis

LCMS data was aligned, peaks picked and isotopes clustered in Genedata Refiner MS (Genedata Expressionist® 11.0.0a, Basel, Switzerland). The subsequent cluster volumes were analysed in Genedata Analyst. A total of 2,734 clusters were identified and cultivars with cannabinoid profiles enriched for total CBD were identified by comparison to 14 cannabinoids (for which standards were available), which were used as input to the clustering (Table 8).

TABLE 8
Cannabinoid standards used for profiling

| Cannabinoid | Formula | Charge | m/z | RT |
|---|---|---|---|---|
| Tetrahydrocannabivarin | C₁₉H₂₆O₂ | 1 | 287.2004 | 7.90 |
| (THCV) | | | | |
| Cannabidivarin (CBDV) | C₁₉H₂₆O₂ | 1 | 287.2006 | 6.45 |
| Cannabinol (CBN) | C₂₁H₂₆O₂ | 1 | 311.2005 | 8.92 |
| Cannabidiol (CBD) | C₂₁H₃₀O₂ | 1 | 315.2316 | 7.65 |
| (−)-Δ9- | C₂₁H₃₀O₂ | 1 | 315.2317 | 9.76 |
| Tetrahydrocannabinol | | | | |
| (THC) | | | | |
| Cannabichromene (CBC) | C₂₁H₃₂O₂ | 1 | 317.2110 | 10.89 |
| Cannabigerol (CBG) | C₂₁H₃₂O₂ | 1 | 317.2473 | 7.52 |
| Tetrahydrocannabivarinic | C₂₀H₂₆O₄ | 1 | 331.1902 | 8.70 |
| acid (THCVA) | | | | |
| Cannabidivarinic acid | C₂₀H₂₆O₄ | 1 | 331.1902 | 6.19 |
| (CBDVA) | | | | |
| Cannabinolic acid (CBNA) | C₂₂H₂₆O₄ | 1 | 355.1901 | 9.73 |
| Cannabidiolic acid (CBDA) | C₂₂H₃₀O₄ | 1 | 359.2214 | 7.16 |
| Cannabichromenic acid | C₂₂H₃₀O₄ | 1 | 359.2216 | 11.18 |
| (CBCA) | | | | |
| Δ9- | C₂₂H₃₀O₄ | 1 | 359.4439 | 10.73 |
| Tetrahydrocannabinolic acid | | | | |
| (THCA) | | | | |
| Cannabigerolic acid (CBGA) | C₂₂H₃₂O₄ | 1 | 361.2371 | 7.41 |

### Terpene Peak Identification Analysis

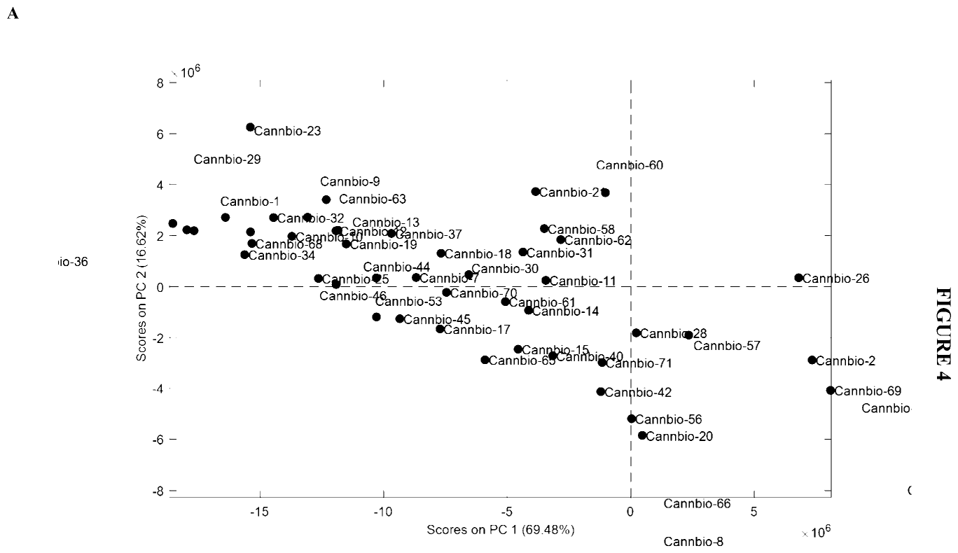

Peak identifications were assigned using MS spectral matching against reference spectra in the NIST/Wiley libraries and Kovats Indicies. Confirmatory identification was done based on retention index, which was calculated for the compounds identified in each sample using an external standard analysed under the same GC conditions. The external standards (Table 9) enabled the assignment of major volatile peaks in the Cannabis strains. Several peaks were not able to be identified with certainty by library matching or by comparison to the standards. These include both putative monoterpenes (M01-M13) and sesquiterpenes (S01-S08). The data was compared with the published values and peak identifications were assigned (Table 9; FIG. 3).

GC-MS data was analysed by PCA using PLSToolbox (Version 8.6.1, Eigenvector Research, Inc.) running on MatLaw (Version R2018a, Mathworks).

TABLE 9
Terpene standards used to identify terpenes in cannabis

| | | | | Retention | |
|---|---|---|---|---|---|
| Peak | RT | | | Index | |
| No. | (min) | Name | m/z | (calculated) | Status |
| 1 | 8.849 | α-Phellandrene | 93.0 | 928 | specID |
| 2 | 9.092 | α-Pinene (+/−) | 93.0 | 937 | Confirmed |
| 3 | 9.590 | Camphene | 93.0 | 955 | Confirmed |
| 4 | 10.383 | β-Pinene (+/−) | 93.0 | 983 | Confirmed |
| 5 | 10.570 | Myrcene | 93.0 | 990 | Confirmed |
| 6 | 11.481 | α-Terpinene | 93.0 | 1021 | Confirmed |
| 7 | 11.848 | Limonene | 68.1 | 1033 | Confirmed |
| 8 | 11.930 | β-Phellandrene | 93.1 | 1036 | specID |
| 9 | 11.983 | Eucalyptol | 81.0 | 1038 | Confirmed |
| 10 | 12.264 | Ocimene isomer | 93.1 | 1047 | Confirmed |
| 11 | 12.700 | γ-Terpinene | 93.1 | 1061 | Confirmed |
| 12 | 13.088 | 4-Thujanol | 93.1 | 1074 | specID |
| 13 | 13.531 | Terpinolene | 93.1 | 1089 | Confirmed |
| 14 | 13.708 | Fenchone | 81.1 | 1095 | Confirmed |
| 15 | 13.868 | Linalool | 93.0 | 1101 | Confirmed |
| 16 | 14.615 | Fenchol | 81.1 | 1126 | specID |
| 17 | 14.844 | Trans-2-Pinanol | 93.1 | 1133 | specID |
| 18 | 16.219 | Borneol | 95.1 | 1180 | Confirmed |
| 19 | 16.835 | α-Terpineol | 93.1 | 1200 | Confirmed |
| 20 | 22.511 | α-Bergamotene | 93.1 | 1406 | specID |
| 21 | 22.854 | β-Bergamotene | 119.1 | 1419 | specID |
| 22 | 23.148 | trans-Caryophyllene | 93.0 | 1431 | Confirmed |
| 23 | 23.280 | γ-Elemene iso1 | 121.0 | 1436 | specID |
| 24 | 23.360 | Bergamontene iso3 | 93.1 | 1439 | specID |
| 25 | 23.467 | α-Guaiene | 105.0 | 1443 | specID |
| 26 | 23.748 | Farnesene | 69.2 | 1454 | Confirmed, |
| | | | | | RI |
| 27 | 24.083 | Humulene | 93.0 | 1467 | specID |
| 28 | 24.780 | (−)-α-Selinene | 105.1 | 1494 | specID |
| 29 | 24.932 | epi-β-selinene | 93.1 | 1500 | specID |
| 30 | 25.050 | sesquiT-coeluting01 | 93.1 | 1510 | specID |
| 31 | 25.174 | δ-Guaiene | 107.0 | 1520 | specID |
| 32 | 25.999 | α-Bisabolene | 93.1 | 1545 | specID |
| 33 | 26.099 | Guaia-3,9-diene | 161.1 | 1549 | specID |
| 34 | 26.210 | 3,7(11)-Selinadiene | 161.1 | 1553 | specID |
| 35 | 26.456 | β-cis-Caryophyllene | 69.2 | 1563 | specID |
| 36 | 26.660 | γ-Elemene iso2 | 121.1 | 1572 | specID |
| 37 | 27.242 | Caryophyllene | 79.0 | 1596 | Confirmed |
| | | oxide | | | |
| 38 | 27.474 | Guaiol | 105.1 | 1606 | Confirmed |
| 39 | 28.187 | β-Cadinene | 189.1 | 1637 | specID |
| 40 | 28.922 | γ-Guriunene | 59.1 | 1669 | specID |
| 41 | 29.081 | sesquiterpene | 107.0 | 1676 | specID |
| 42 | 29.455 | α-Bisabolol | 93.0 | 1692 | Confirmed |

### Quantitation

LCMS chromatograms were processed using Thermo LCQuan v.2.7 software by extracted ion using the m/z values specified in Table 7 with a window of 5 ppm or by PDA analysis at 280 nm. Calibration curves were developed using the serial diluted standards and the amount of each cannabinoid in the cultivars calculated.

GC-MS chromatograms were processed using Agilent MassHunter software using the retention time and m/z profiles of the standards specified in Table 4.

### F. Genomic Analysis

Whole genome resequencing for 23 Cannabis plants was performed using Illumina-based short read sequencing (2×150 bp) to an expected depth of 10× genome coverage for all samples. The publicly available reference genome sequence of Purple Kush (version—canSat3) was used to align all genome sequences from all samples. Following genome alignment with the software package BWA, using the MEM algorithm variant sites were called using the software package samtools. The list of variant sites was then extensively filtered for quality, based on <50% missing data, >5% minor allele frequency within the data set used, as well as only selecting variant sites that were biallelic, and that were not indels. The filtering process was performed using the software packages bcftools and vcftools. A total of 2,773,425 variant sites remained after filtering.

### G. Supercritical Fluid Extraction (SCE) of Cannabinoids

Extract comprising cannabinoids were prepared from air dried and cured mature plant material using supercritical fluid extraction (SCE) with CO2, as previously described in Khaw et al. (Molecules, 2017, 22:1186). Briefly, cured biomass was extracted using SFE with CO2 using the following parameters:
Temperature of 60° C.;
Flow rate of 150 g/min; and
Pressure of 150 bar.

### Results

### Cannabinoid Chemotyping

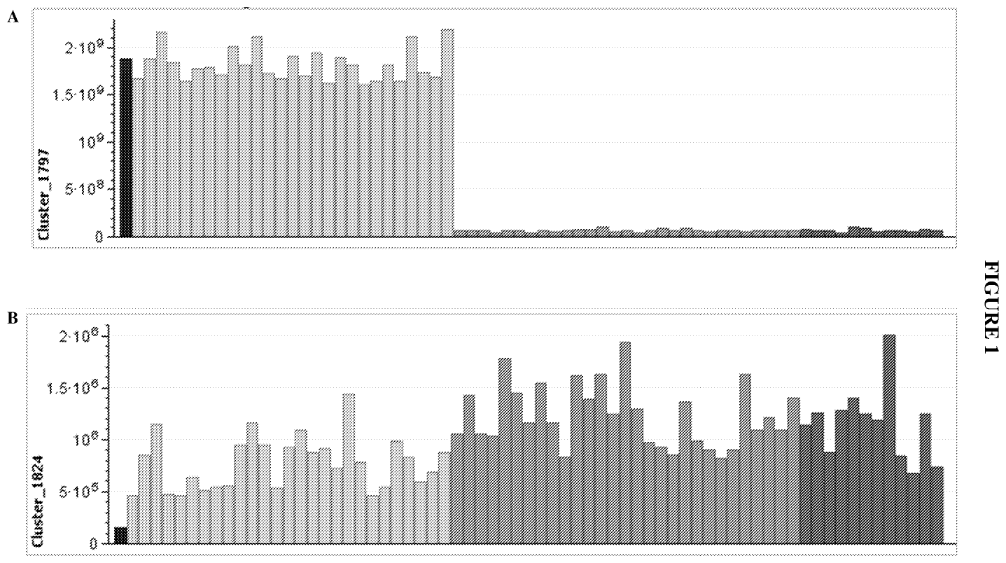

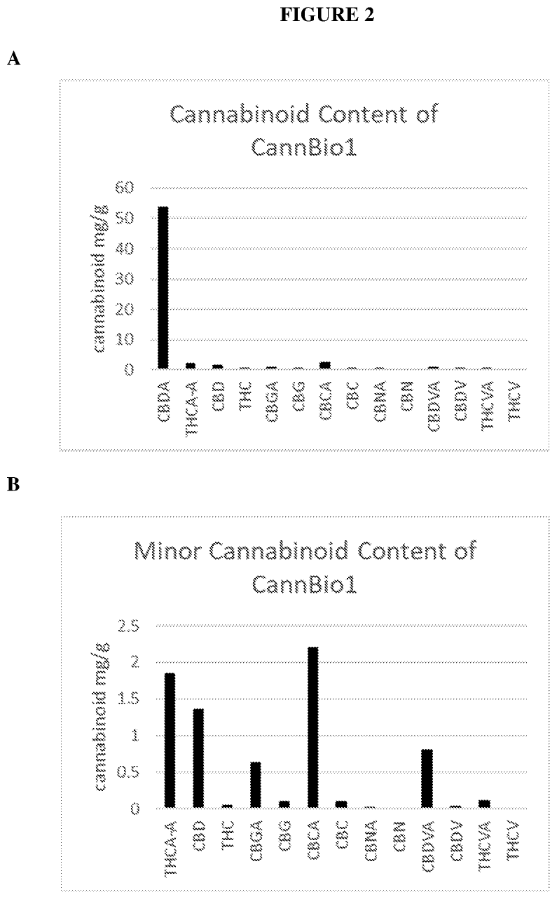

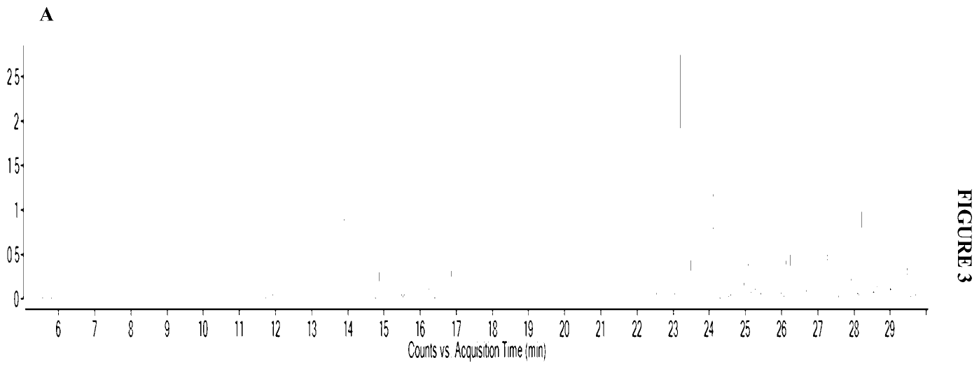

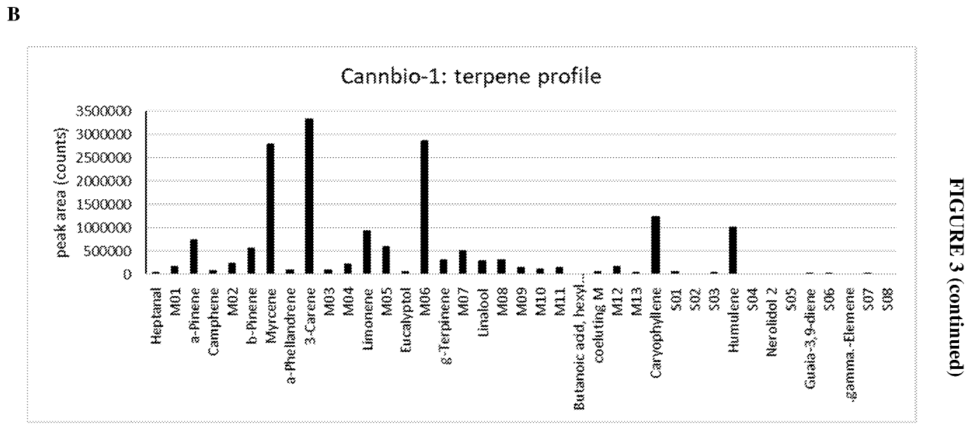

LCMS analysis was undertaken to identify plants with cannabinoid profiles enriched for total CBD. For untargeted analysis the intensity cut off was stringent, meaning only peaks that were relatively intense would be selected. Post peak alignment and isotope clustering a total of 2,734 isotope clusters were identified in the combined dataset. Since standards were run under the same conditions along with the plant extracts, cannabinoid profiles were generated corresponding to the known cannabinoids (Table 8). Enrichment for CBDA (FIG. 1A) and THCA (FIG. 2A) was used as an initial comparator to group the Cannabis plants. For this analysis, the plant material had not been heated so the acid forms were present at higher levels than the respective neutral species (Citti et al. 2018, Phytochemical Analysis, 29: 539-48).

Hierarchical cluster analysis of both the entire data set and the 14 cannabinoids identified a Cannabis strains with a cannabinoid profile enriched for total CBD, which also had relatively low levels of total THC (i.e., THC+THCA) (FIG. 1).

### Quantitative Analysis

To fully describe the cannabinoid profile enriched for total CBD, quantitative analysis was performed on CannBio-1 (FIG. 2).

The results obtained from this analysis indicated that the total CBD is 55.10 mg/g on a dried weight basis. Total THC is 1.89 mg/g. The other cannabinoids are also present in low levels: total CBG is 0.71 mg/g, total CBC is 2.29 mg/g, total CBN is 0.02 mg/g, total CBDV is 0.83 mg/g and total THCV is 0.10 mg/g. The results from this analysis are summarised in Table 10, below.

TABLE 10
Quantitative analysis of cannabinoids in CBD-enriched cannabis

| | | | % of total |
|---|---|---|---|
| | Concentration | Ratio | cannabinoid |
| Cannabinoid | (mg/g) | (CBD:Cannabinoid) | content |
| CBD | 55.1 | 1 | 90.42 |
| THC | 1.89 | 29.15 | 3.10 |
| CBG | 0.71 | 77.61 | 1.17 |
| CBC | 2.29 | 24.06 | 3.76 |
| CBN | 0.02 | 2755 | 0.03 |
| CBDV | 0.83 | 66.39 | 1.36 |
| THCV | 0.1 | 551 | 0.16 |
| TOTAL | 60.94 | | |

### Genomic Analysis

Whole genome resequencing was performed for 23 Cannabis plants comprising a cannabinoid profile enriched for total CBD. A total of 2,773,425 variant sites remained after filtering. Allelic frequencies for each of the defined groups for all variant sites were then generated from the genotypic data. From each cluster, only sites that had genotypic frequencies that were either 100% or 0% for the reference allele were then identified. The four lists of variant sites were then merged and only variant sites that appeared in all four lists were retained. A total of 186 variant sites remained that were capable of distinguishing Cannabis plants with a cannabinoid profile enriched for total CBD from Cannabis plants with other cannabinoid profiles (Table 2). In all cases the base called in the expanded set of samples with a cannabinoid profile enriched for total CBD genotyped at 100% frequency as the alternative allele, whilst all other clusters genotyped as the reference nucleotide.

### Terpene Profile

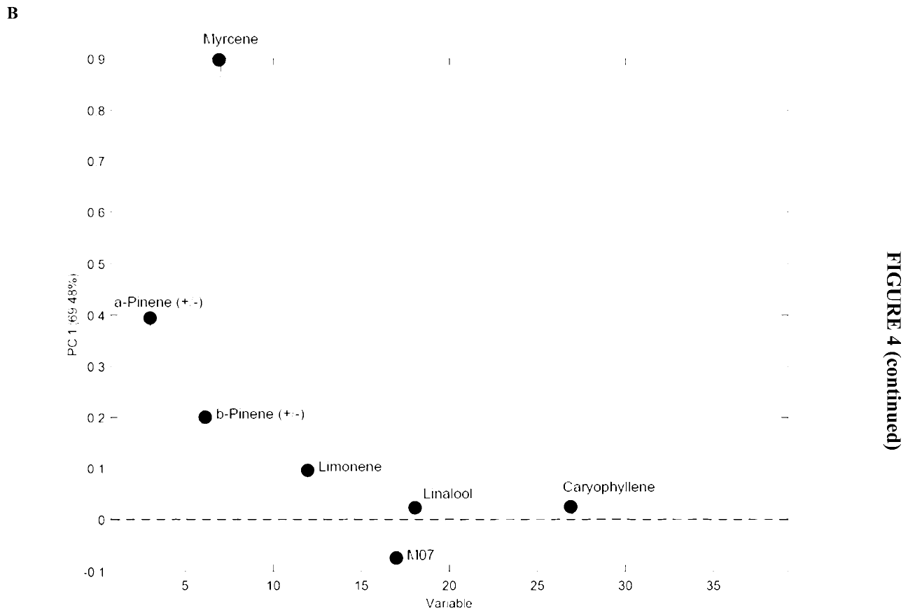

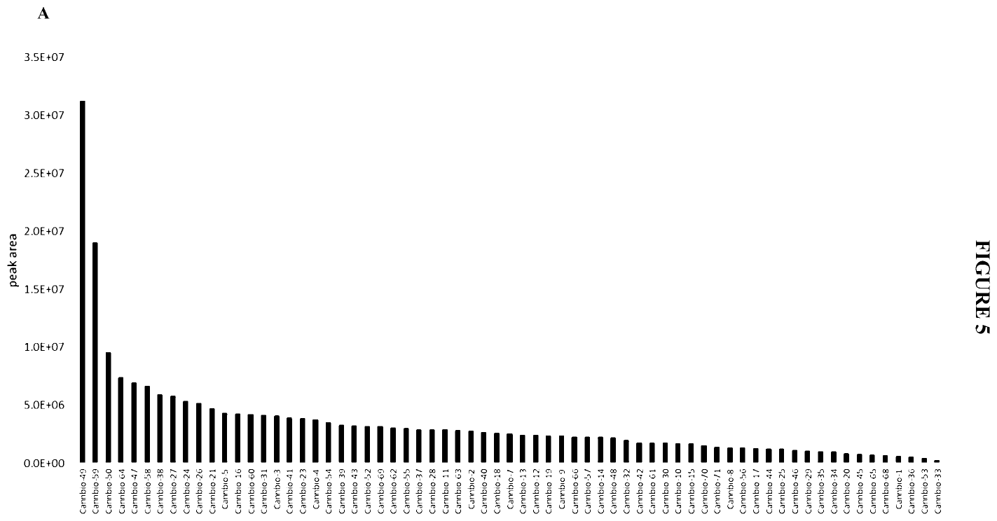

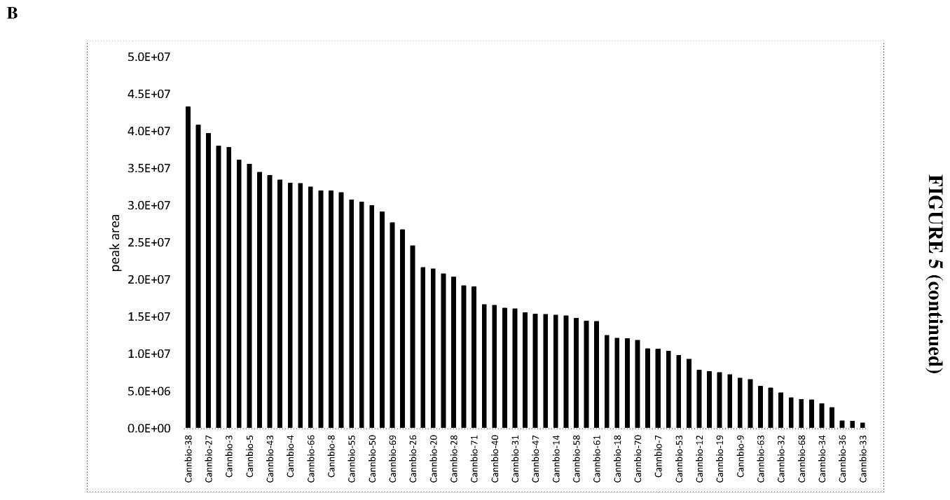

To further define the chemotype of the Cannabis plants, terpene profiles were evaluated using GC-MS. Using Principal Component Analysis (PCA), PC1 explained 69.48% of variance, and PC2 explained 16.62% of variance in the data (total 86.1%) (FIG. 4A). PC1 is characterised by plants enriched for myrcene, i.e., myrcene-enriched (FIG. 4B). The abundance of myrcene varied between the different Cannabis strains (FIG. 5B). The abundance of β-pinene was also quantified for comparative analysis (FIG. 5A).

In plants identified as comprising a cannabinoid profile enriched for total CBD, the abundance of myrcene and β-pinene was determined to according to peak area (FIG. 5B). The relative abundance (ratio) of myrcene to β-pinene in these Cannabis strains was about 5:1.

### CONCLUSION

The quantitative analysis of an extract taken from the Cannabis plant identified as having a CBD-enriched cannabinoid profile confirmed that this plant is characterised by high levels of CBD and relatively low levels of THC, and therefore would be suitable for treatment of conditions where CBD is likely to provide a therapeutic benefit. The CBD-enriched chemotypic profile of the Cannabis plant was associated with a unique genomic profile of 186 allelic variants shared by 23 additional Cannabis plants comprising a CBD-enriched cannabinoid profile.

These genomic features further distinguish these new, CBD-enriched Cannabis varieties from other Cannabis varieties. Surprisingly, the allelic variants identified by this analysis were not located in the genomic scaffolds containing genes involved in cannabinoid biosynthesis, as identified in the reference genome (Table 11). These results indicate that the unique SNP profile of the Cannabis plants described herein, comprising a CBD-enriched cannabinoid profile, have a genomic profile that is distinguished from other known Cannabis varieties, particularly those with THC-enriched cannabinoid profiles such as Purple Kush.

TABLE 11
Cannabinoid pathway genes in canSat3 assembly

| | |
|---|---|
| Gene | Scaffold |
| THCAS | scaffold19603 |
| Desaturase | scaffold71447 |
| LOX1 | scaffold53609 (part 1), scaffold12507 (part 2) |
| HPL | scaffold14797 (part 1), C31982282 (part 2), |
| | scaffold21904 (part 3) |
| AAE1 | scaffold1750 (1^{st} copy, full), scaffold29030 (2^{nd} |
| | copy, part 1), scaffold83207 (2^{nd} copy, part 2) |
| AAE3 | scaffold104648 (Original), scaffold 108682 (Copy, |
| | processed) |
| OLS | scaffold15717 (1^{st} copy), scaffold16618 (2^{nd} copy) |
| PT | scaffold105794 (part 1), C31963486 (part 2), |
| | scaffold73228 (part 4) |
| DXS | scaffold19453 (part 1), scaffold49473 (part 2), |
| | scaffold34324 (part 3) |
| DXR | scaffold44357 (1^{st} copy), scaffold26322 (2^{nd} copy) |

### Comparative Analysis

The chemotypic features of these new, CBD-enriched Cannabis varieties may be used to distinguish CBD-enriched Cannabis varieties from other Cannabis varieties.

Cannabis Plants with a Cannabinoid Profile Enriched for Total CBD and Total THC

Quantitative analysis was performed on the 27 Cannabis strains with a cannabinoid profile enriched for total CBD and total THC. The results obtained from this analysis are provided in Table 12, below (mg/g).

TABLE 12
Quantitative analysis of cannabinoids in CBD-and THC-enriched cannabis

| | | | | | | | | Total |
|---|---|---|---|---|---|---|---|---|
| Strain # | CBD | THC | CBG | CBC | CBN | CBDV | THCV | cannabinoid |
| 2 | 53.33 | 33.96 | 1.21 | 3.12 | 0.1 | 0.23 | 0.24 | 92.19 |
| 3 | 91.42 | 57.2 | 2.52 | 5.39 | 0.05 | 0.2 | 0.32 | 157.1 |
| 6 | 55.49 | 31.36 | 2.38 | 3.08 | 0.09 | 0.3 | 0.35 | 93.05 |
| 7 | 69.26 | 36.69 | 2.36 | 4.01 | 0.11 | 0.33 | 0.29 | 113.05 |
| 8 | 74.14 | 29.76 | 3.64 | 4.39 | 0.15 | 0.37 | 0.34 | 112.79 |
| 9 | 69.51 | 33.38 | 2.55 | 3.77 | 0.13 | 0.32 | 0.35 | 110.01 |
| 10 | 51.97 | 22.68 | 2.11 | 2.71 | 0.09 | 0.29 | 0.26 | 80.11 |
| 11 | 65.71 | 35.09 | 2.24 | 3.46 | 0.09 | 0.32 | 0.29 | 107.2 |
| 12 | 70.87 | 33.14 | 3.72 | 3.99 | 0.1 | 0.37 | 0.36 | 112.55 |
| 13 | 64.27 | 30.26 | 1.9 | 3.04 | 0.13 | 0.35 | 0.32 | 100.27 |
| 14 | 78.37 | 41.58 | 4.48 | 3.94 | 0.16 | 0.41 | 0.42 | 129.36 |
| 15 | 73.06 | 38.33 | 2.37 | 3.77 | 0.07 | 0.39 | 0.45 | 118.44 |
| 16 | 96.97 | 74.48 | 5.12 | 5.22 | 0.13 | 0.47 | 0.49 | 182.88 |
| 17 | 76.72 | 36.42 | 2.86 | 4.05 | 0.1 | 0.37 | 0.31 | 120.83 |
| 18 | 67.57 | 22.29 | 2.14 | 3.7 | 0.08 | 0.41 | 0.41 | 96.6 |
| 19 | 76.61 | 37.91 | 3.64 | 4.75 | 0.1 | 0.39 | 0.35 | 123.75 |
| 20 | 86.25 | 36.4 | 3.13 | 5.07 | 0.11 | 0.43 | 0.54 | 131.93 |
| 21 | 56.72 | 20.86 | 1.06 | 3.07 | 0.08 | 0.1 | 0.27 | 82.16 |
| 22 | 68.15 | 25.38 | 1.17 | 4.12 | 0.11 | 0.12 | 0.29 | 99.34 |
| 23 | 51.19 | 20.49 | 1.9 | 3.16 | 0.09 | 0.09 | 0.16 | 77.08 |
| 24 | 74.74 | 27.35 | 1.26 | 4.24 | 0.1 | 0.14 | 0.27 | 108.1 |
| 25 | 73.92 | 38.55 | 1.95 | 4.27 | 0.12 | 0.13 | 0.25 | 119.19 |
| 26 | 82.46 | 43.34 | 1.46 | 6.21 | 0.11 | 0.2 | 0.27 | 134.05 |
| 27 | 70.43 | 50.77 | 2.77 | 4.04 | 0.08 | 0.41 | 0.33 | 128.83 |
| 28 | 65.4 | 33.14 | 0.94 | 3.41 | 0.19 | 0.31 | 0.35 | 103.74 |
| 29 | 43.1 | 22.39 | 1.17 | 2.56 | 0.11 | 0.2 | 0.21 | 69.74 |
| 30 | 42.82 | 28.36 | 1.3 | 2.22 | 0.12 | 0.23 | 0.28 | 75.33 |

In plants identified as comprising a cannabinoid profile enriched for total CBD and total THC, the abundance of myrcene and β-pinene was determined according to peak area (FIG. 5). The relative abundance (ratio) of myrcene to β-pinene in these Cannabis strains was determined to be from about 40:1 and about 1:1.

Cannabis Plants with a Cannabinoid Profile Enriched for Total THC and Total CBG

Quantitative analysis was performed on the 29 Cannabis strains with a cannabinoid profile enriched for total THC and CBG. The results obtained from this analysis are provided in Table 13, below.

TABLE 13
Quantitative analysis of cannabinoids in THC- and CBG- enriched cannabis

| | | | | | | | | Total |
|---|---|---|---|---|---|---|---|---|
| Cannabis | | | | | | | | cannabiniod |
| strain # | CBD | THC | CBG | CBC | CBN | CBDV | THCV | (mg/g) |
| 31 | 0.55 | 80.74 | 4.82 | 2.94 | 0.16 | 0 | 0.27 | 89.48 |
| 32 | 0.51 | 110.11 | 6.15 | 3.51 | 0.2 | 0 | 0.24 | 120.72 |
| 33 | 0.37 | 66.23 | 2.31 | 3.85 | 0.29 | 0 | 0.13 | 73.18 |
| 34 | 0.68 | 84.22 | 2.33 | 3.45 | 0.26 | 0 | 0.16 | 91.1 |
| 35 | 0.52 | 76.54 | 2.04 | 4.02 | 0.23 | 0 | 0.19 | 83.54 |
| 36 | 0.27 | 66.44 | 3.99 | 3.38 | 0.17 | 0 | 0.3 | 74.55 |
| 37 | 0.99 | 119.9 | 5.39 | 4.85 | 0.14 | 0 | 0.66 | 131.93 |
| 38 | 0.7 | 134.54 | 6.8 | 3.28 | 0.21 | 0 | 0.75 | 146.28 |
| 39 | 0.41 | 134.24 | 6.23 | 2.67 | 0.18 | 0 | 0.93 | 144.66 |
| 40 | 0.46 | 119.75 | 8.98 | 2.9 | 0.24 | 0 | 0.91 | 133.24 |
| 41 | 0.38 | 99.17 | 4.54 | 1.6 | 0.18 | 0 | 0.49 | 106.36 |
| 42 | 0.55 | 93.37 | 4.34 | 0.88 | 0.21 | 0 | 0.62 | 99.97 |
| 43 | 0.38 | 129.29 | 8.28 | 4.89 | 0.17 | 0 | 1.41 | 144.42 |
| 44 | 0.34 | 105.7 | 3.53 | 1.62 | 0.15 | 0 | 0.78 | 112.12 |
| 45 | 0.25 | 71.53 | 2.23 | 1.65 | 0.21 | 0 | 0.29 | 76.16 |
| 46 | 0.36 | 81.72 | 1.67 | 1.1 | 0.18 | 0 | 0.38 | 85.41 |
| 47 | 0.39 | 124.4 | 3.49 | 2.97 | 0.23 | 0 | 0.71 | 132.19 |
| 48 | 0.41 | 115.05 | 4.87 | 2.26 | 0.17 | 0 | 0.69 | 123.45 |
| 49 | 1.05 | 146.94 | 4.26 | 3.6 | 0.25 | 0 | 1.2 | 157.3 |
| 50 | 0.61 | 142.55 | 7.59 | 3.31 | 0.26 | 0 | 1.02 | 155.34 |
| 51 | 0.42 | 123.04 | 4.37 | 1.53 | 0.32 | 0 | 1.12 | 130.8 |
| 52 | 0.62 | 134.96 | 9.7 | 1.58 | 0.21 | 0 | 0.85 | 147.92 |
| 53 | 0.35 | 79.75 | 1.8 | 1.07 | 0.18 | 0 | 0.51 | 83.66 |
| 54 | 0.54 | 103.51 | 6.01 | 1.81 | 0.09 | 0 | 0.52 | 112.48 |
| 55 | 0.46 | 116.04 | 5.28 | 1.73 | 0.13 | 0 | 0.75 | 124.39 |
| 56 | 0.49 | 91.75 | 4.56 | 0.97 | 0.13 | 0 | 0.47 | 98.37 |
| 57 | 0.49 | 114.39 | 4.47 | 1.38 | 0.14 | 0 | 0.76 | 121.63 |
| 58 | 0.5 | 132.04 | 7.74 | 1.69 | 0.11 | 0 | 0.67 | 142.75 |
| 59 | 0.77 | 203.58 | 4.81 | 1.98 | 0.21 | 0 | 0.95 | 212.3 |

In plants identified as comprising a cannabinoid profile enriched for total THC and total CBG, the abundance of myrcene and β-pinene was determined according to peak area (FIG. 5). The relative abundance (ratio) of myrcene to β-pinene in these Cannabis strains was determined to be from about 60:1 and 1:1.

Cannabis Plants with a Cannabinoid Profile Enriched for Total THC, Total CBG and Total THCV

Quantitative analysis was performed on the 12 Cannabis strains with a cannabinoid profile enriched for total THC, CBG and THCV. The results obtained from this analysis are provided in Table 14, below.

TABLE 14
Quantitative analysis of cannabinoids in THC/CBG/THCV-enriched cannabis

| | | | | | | | | Total |
|---|---|---|---|---|---|---|---|---|
| Cannabis | | | | | | | | cannabinoid |
| strain # | CBD | THC | CBG | CBC | CBN | CBDV | THCV | (mg/g) |
| 60 | 0.73 | 124.09 | 1.83 | 1.77 | 0.21 | 0.02 | 5.47 | 134.12 |
| 61 | 0.68 | 96.31 | 2.74 | 2.29 | 0.3 | 0.02 | 4.54 | 106.88 |
| 62 | 0.46 | 120.87 | 5.3 | 2.13 | 0.24 | 0.02 | 3.26 | 132.28 |
| 63 | 0.8 | 104.57 | 8.89 | 1.94 | 0.18 | 0.03 | 6.57 | 122.98 |
| 64 | 0.42 | 139.17 | 4.47 | 2.32 | 0.2 | 0.02 | 5.63 | 152.23 |
| 65 | 0.29 | 85.67 | 1.47 | 1.21 | 0.12 | 0.02 | 2.74 | 91.52 |
| 66 | 0.66 | 99.73 | 1.37 | 1.32 | 0.12 | 0.02 | 2.79 | 106.01 |
| 67 | 0.61 | 86.69 | 1.5 | 0.99 | 0.14 | 0.02 | 3.12 | 93.07 |
| 68 | 0.38 | 96.7 | 2.08 | 5.74 | 0.25 | 0.02 | 2.68 | 107.85 |
| 69 | 0.5 | 81.06 | 3.08 | 1.58 | 0.22 | 0.02 | 2.3 | 88.76 |
| 70 | 0.27 | 78.86 | 1.5 | 3.23 | 0.3 | 0.02 | 2.23 | 86.41 |
| 71 | 0.45 | 83.39 | 2.01 | 1.97 | 0.26 | 0.02 | 2.49 | 90.59 |

In plants identified as comprising a cannabinoid profile enriched for total THC and total CBG, the abundance of myrcene and β-pinene was determined according to peak area (FIG. 5). The relative abundance (ratio) of myrcene to β-pinene in these Cannabis strains was determined to be from about 50:1 and 2.5:1.

1. A Cannabis plant, or a part thereof, comprising a cannabinoid profile enriched for total CBD, wherein the cannabinoid profile comprises a level of total CBD and a level of total THC at a ratio of from about 10:1 to about 50:1 (CBD:THC), wherein the total CBD comprises cannabidiol (CBD) and cannabidiolic acid (CBDA), and the total THC comprises Δ-9-tetrahydrocannabinol (THC) and Δ-9-tetrahydrocannabinolic acid (THCA); wherein the level of total CBD is greater than the level of a reference cannabinoid selected from the group consisting of:
(a) total CBC, wherein the total CBC comprises cannabichromene (CBC) and cannabichromene acid (CBCA);
(b) total CBG, wherein the total CBG comprises cannabigerol (CBG) and cannabigerolic acid (CBGA);
(c) total CBN, wherein the total CBN comprises cannabinol (CBN) and cannabinolic acid (CBNA);
(d) total THCV, wherein the total THCV comprises tetrahydrocannabivarin (THCV) and tetrahydrocannabivarinic acid (THCVA); and
(e) total CBDV, wherein the total CBDV comprises cannabidivarin (CBDV) and cannabidivarinic acid (CBDVA),
and wherein the Cannabis plant comprises a nucleic acid sequence that encodes a wild-type THCA synthase.

2. The Cannabis plant of claim 1, or a part thereof, wherein the part is a female inflorescence.

3. The Cannabis plant of claim 1, or a part thereof, wherein:
(a) the level of total CBD is at least 80% by weight of the total cannabinoid content of the dry weight of plant material; and/or
(b) the level of total THC is from about 1% to about 10% by weight of the total cannabinoid content of the dry weight of plant material.

4. (canceled)

5. The Cannabis plant of claim 1, or a part thereof, wherein the reference cannabinoid is:
(a) total CBC, optionally wherein:
(i) the level of total CBD is present at a ratio of from about 10:1 to about 50:1 to the level of total CBC (CBD:CBC); and/or
(ii) the level of total CBC is from about 1% to about 10% by weight of the total cannabinoid content of the dry weight of plant material;
(b) total CBG, optionally wherein:
(i) the level of total CBD is present at a ratio of from about 10:1 to about 100:1 to the level of total CBG (CBD:CBG); and/or
(ii) the level of total CBG is from about 1% to about 5% by weight of the total cannabinoid content of the dry weight of plant material;
(c) total CBN, optionally wherein:
(i) the level of total CBD is present at a ratio of from about 2000:1 to about 3000:1 to the level of total CBN (CBD:CBN); and/or
(ii) the level of total CBN is from about 0.01% to about 1% by weight of the total cannabinoid content of the dry weight of plant material;
(d) total CBDV, optionally wherein:
(i) the level of total CBD is present at a ratio of from about 10:1 to about 80:1 to the level of total CBDV (CBD:CBDV); and/or
(ii) the level of total CBDV is from about 1% to about 10% by weight of the total cannabinoid content of the dry weight of plant material; or
(e) total THCV, optionally wherein:
(i) the level of total THCV is present at a ratio of from about 400:1 to about 700:1 of the level of total THCV (CBD:THCV); and/or
(ii) the level of total THCV in the plant material is from about 0.05% to about 1% by weight of the total cannabinoid content of the dry weight of plant material.

6-19. (canceled)

20. The Cannabis plant of claim 1, or a part thereof, comprising one or more SNP(s) selected from the group consisting of Variant Nos: 1-186.

21. The Cannabis plant of claim 1, or a part thereof, comprising one or more terpenes selected from the group consisting of α-phellandrene, α-pinene, camphene, β-pinene, myrcene, limonene, eucalyptol, γ-terpinene, linalool, γ-elemene, humulene, nerolidol, guaia-3,9-diene and caryophyllene, preferably comprising one or more terpenes selected from the group consisting of myrcene and β-pinene.

22. (canceled)

23. The Cannabis plant of claim 21, or a part thereof, wherein the level of myrcene is present at a ratio of about 5:1 to the level of β-pinene.

24-37. (canceled)

38. A method of producing an extract comprising cannabinoids from a Cannabis plant, the method comprising the steps of:
(a) harvesting plant material from the Cannabis plant of claim 1;
(b) at least partially drying the harvested plant material of step (a); and
(c) extracting cannabinoids from the at least partially dried plant material of step (b), thereby producing an extract comprising cannabinoids, optionally
wherein the extract comprises a level of total CBD and a level of total THC at a ratio of from about 10:1 to about 50:1 (CBD:THC), and wherein the level of total CBD is greater than the level of a reference cannabinoid selected from the group consisting of total CBC, total CBG, total CBN, total THCV, and total CBDV, or
wherein the extract comprises total CBD, total THC, and one or more minor cannabinoids selected from the group consisting of total CBC, total CBG, total CBN, total THCV, total CBDV, total CBL, and total A8-THC, wherein the total CBD and the total THC are present in the extract at a ratio of from about 10:1 to about 50:1 (CBD:THC), and wherein the one or more minor cannabinoids is present in the extract in an amount of from about 0.01% to about 10% by weight of the total cannabinoid content of the extracted cannabinoids.

39-40. (canceled)

41. The method of claim 38, wherein the plant material comprises female inflorescence.

42. The method of claim 38, wherein cannabinoids are extracted from the at least partially dried plant material of step (b) by supercritical fluid extraction.

43. (canceled)

44. The Cannabis plant of claim 1, which is an extract comprising a cannabinoid profile enriched for total CBD, wherein the cannabinoid profile comprises a level of total CBD and a level of total THC at a ratio of from about 10:1 to about 50:1 (CBD:THC), and wherein the level of total CBD is greater than the level of a reference cannabinoid selected from the group consisting of total CBC, total CBG, total CBN, total THCV, and total CBDV.

45. The Cannabis plant of claim 1, which is a total CBD-enriched cannabinoid extract comprising total CBD, total THC, and one or more minor cannabinoids selected from the group consisting of: total CBC, total CBG, total CBN, total THCV, total CBDV, total CBL, and total Δ8-THC, wherein the total CBD and the total THC are present in the extract at a ratio of from about 10:1 to about 50:1 (CBD:THC); and wherein the one or more minor cannabinoids is present in the extract in an amount of from about 0.01% to about 10% by weight of the total cannabinoid content of the extract.

46. A method for selecting a Cannabis plant comprising a cannabinoid profile enriched for total CBD from a plurality of different Cannabis plants, the method comprising:
(a) harvesting plant material from a plurality of different Cannabis plants;
(b) at least partially drying the harvested plant material of step (a);
(c) measuring in the at least partially dried plant material of step (b) a level of total CBD, total THC and one or more reference cannabinoids selected from the group consisting of THCV, CBDV, CBN, CBC, CBG, THCVA, CBDVA, CBNA, CBCA, and CBGA to generate a cannabinoid profile for each of the plurality of Cannabis plants;
(d) optionally, measuring in the at least partially dried plant material of step (b) one or more terpenes selected from the group consisting of α-phellandrene, α-pinene, camphene, β-pinene, myrcene, limonene, eucalyptol, γ-terpinene, linalool, γ-elemene, humulene, nerolidol, guaia-3,9-diene and caryophyllene, preferably myrcene and β-pinene, to generate a terpene profile for each of the plurality of Cannabis plants; and
(e) on the basis of the measurements from step (c) and optionally, step (d), selecting from the plurality of different Cannabis plants a Cannabis plant comprising cannabinoid profile enriched for total CBD and, comprising a level of total CBD and a level of total THC at a ratio of from about 10:1 to about 50:1 (CBD:THC), wherein the total CBD comprises CBD and CBDA, and the total THC comprises THC and THCA; wherein the level of total CBD is greater than the level of a reference cannabinoid selected from the group consisting of:
(i) total CBC, wherein the total CBC comprises CBC and CBCA;
(ii) total CBG, wherein the total CBG comprises CBG and CBGA;
(iii) total CBN, wherein the total CBN comprises CBN and CBNA;
(iv) total THCV, wherein the total THCV comprises THCV and THCVA; and
(v) total CBDV, wherein the total CBDV comprises CBDV and CBDVA.

47. (canceled)

48. The method of claim 46, wherein the plant material comprises female inflorescence.

49. The method of claim 46, wherein:
(a) the level of total CBD is at least 80% by weight of the total cannabinoid content of the at least partially dried weight of the plant material; and/or
(b) the level of total THC is from about 1% to about 10% by weight of the total cannabinoid content of the at least partially dried weight of the plant material.

50. (canceled)

51. The method of claim 46, wherein the reference cannabinoid is:
(a) total CBC, optionally wherein:
(i) the level of total CBD is present at a ratio of from about 10:1 to about 50:1 to the level of total CBC (CBD:CBC) in the plant material; and/or
(ii) the level of total CBC is from about 1% to about 10% by weight of the total cannabinoid content of the at least partially dried weight of the plant material;
(b) total CBG, optionally wherein:
(i) the level of total CBD is present at a ratio of from about 10:1 to about 100:1 to the level of total CBG (CBD:CBG) in the plant material; and/or
(ii) the level of total CBG is from about 1% to about 5% by weight of the total cannabinoid content of the at least partially dried weight of the plant material;
(c) total CBN, optionally wherein:
(i) the level of total CBD is present at a ratio of from about 2000:1 to about 3000:1 to the level of total CBN (CBD:CBN) in the plant material; and/or
(ii) the level of total CBN is from about 0.01% to about 1% by weight of the total cannabinoid content of the at least partially dried weight of the plant material;
(d) total CBDV, optionally wherein:
(i) the level of total CBD is present at a ratio of from about 10:1 to about 80:1 to the level of total CBDV (CBD:CBDV) in the plant material; and/or
(ii) the level of total CBDV is from about 1% to about 10% by weight of the total cannabinoid content of the at least partially dried weight of the plant material; or
(e) total THCV, optionally wherein:
(i) the level of total THCV is present at a ratio of from about 400:1 to about 700:1 of the level of total THCV (CBD:THCV) in the plant material; and/or
(ii) the level of total THCV in the plant material is from about 0.05% to about 1% by weight of the total cannabinoid content of the at least partially dried weight of the plant material.

52-65. (canceled)

66. The method of claim 46, wherein the plant material comprises one or more SNP(s) selected from the group consisting of Variant Nos: 1-186.

67-68. (canceled)

69. The method of claim 46, wherein the level of myrcene is present at a ratio of about 5:1 to the level of β-pinene in the plant material.

70. The method of claim 46, wherein the selected Cannabis plant is crossed with a different Cannabis plant to produce an F1 hybrid.

71. The method of claim 46, wherein regenerable cells isolated from the selected Cannabis plant are:
a) transformed with a heterologous nucleic acid sequence and cultured for a time and under conditions suitable to produce a transgenic Cannabis plant; or
(b) transfected with a gene editing construct comprising a nucleic acid sequence encoding a DNA-recognition moiety and cultured for a time and under conditions suitable to produce a non-transgenic Cannabis plant with modified gene expression.

72. (canceled)
